(19) United States
(12) Patent Application Publication
(10) Pub. No.: US 2023/0101618 A1
(43) Pub. Date: Mar. 30, 2023

(54) PREDICTIVE OUTCOME PROFILING FOR USE OF AN ANTI-SEMAPHORIN-4D BINDING MOLECULE TO TREAT NEURODEGENERATIVE DISORDERS

(71) Applicant: VACCINEX, INC., Rochester, NY (US)

(72) Inventors: Maurice Zauderer, Pittsford, NY (US); Elizabeth Evans, Bloomfield, NY (US); Terrence Fisher, Rochester, NY (US)

(21) Appl. No.: 17/934,660

(22) Filed: Sep. 23, 2022

Related U.S. Application Data
(60) Provisional application No. 63/248,663, filed on Sep. 27, 2021.

(30) Foreign Application Priority Data
Sep. 27, 2021 (US) PCT/US2021/052142

(51) Int. Cl. C07K 16/28; A61P 25/28

(52) U.S. Cl. CPC C07K 16/2803; A61P 25/28

(57) ABSTRACT

Provided herein are methods for selecting subjects having a neurodegenerative disorder for treatment with an isolated binding molecule which specifically binds to semaphorin-4D (SEMA4D), method for predicting the treatment outcome of subjects having a neurodegenerative disorder that are treated with an isolated binding molecule which specifically binds to SEMA4D and methods for treating subjects having a neurodegenerative disorder with an isolated binding molecule which specifically binds to SEMA4D.

### CROSS-REFERENCE TO RELATED APPLICATIONS

This is a non-provisional of and claims the benefit priority under 35 U.S.C. § 119(e) to U.S. Provisional Patent Application No. 63/248,663, filed on Sep. 27, 2021, and International Application No. PCT/US2021/052142, filed Sep. 27, 2021, the entirety of which applications are incorporated by reference herein.

### REFERENCE TO SEQUENCE LISTING SUBMITTED ELECTRONICALLY

The instant application contains a Sequence Listing which has been submitted electronically in XMLASCII format and is hereby incorporated by reference in its entirety. Said XMLASCII copy, created on Oct. 24, 2022Sep. 27, 2021, is named 8555_040_SL and is 55,20554.2KB bytes in size.

### BACKGROUND OF THE DISCLOSURE

Semaphorin 4D (SEMA4D), also known as CD100, is a transmembrane protein (e.g., SEQ ID NO: 1 (human); SEQ ID NO: 2 (murine)) that belongs to the semaphorin gene family. SEMA4D is expressed on the cell surface as a homodimer, but upon cell activation SEMA4D can be released from the cell surface via proteolytic cleavage to generate sSEMA4D, a soluble form of the protein, which is also biologically active. See Suzuki et al., Nature Rev. Immunol. 3:159-167 (2003); Kikutani et al., Nature Immunol. 9:17-23 (2008). SEMA4D has been implicated in the development of neurodegenerative disorders, autoimmune diseases, demyelinating diseases, and certain cancers.

SEMA4D has been implicated in the development of neurodegenerative disorders, autoimmune diseases, demyelinating diseases, and certain cancers. However, the effect of blocking SEMA4D signaling on the organization and function of the central nervous system (CNS) including brain and spinal cord and on behaviors controlled by the CNS remains to be elucidated. This is important because changes in the CNS have a profound influence on a subject's behavior and quality of life. In particular, such changes can impact a subject's neuropsychiatric behavior, cognitive behavior, and motor skills.

Neurodegenerative and neuroinflammatory disorders are recognized as a highly heterogeneous diseases that develop from diverse causes and progression of the disorder is highly likely to have an effect on treatment outcome. Thus, there is a need to help predict response to treatment for patients with neurodegenerative and neuroinflammatory disorders.

### BRIEF SUMMARY OF THE DISCLOSURE

Methods for selecting a subject having, determined to have, or suspected of having a neurodegenerative disorder for treatment with an isolated antibody or antigen-binding fragment thereof that specifically binds to semaphorin-4D (SEMA4D) are provided. The methods comprise (i) determining the cognitive and/or functional impairment assessment score for the subject in one or more standard cognitive and/or functional assessment tests; and (ii) selecting the subject for treatment if the score satisfies a predetermined value indicative of mild cognitive impairment (MCI), mild dementia, moderate cognitive impairment, or Stage I or Stage II Huntington's disease. In certain embodiments, the one or more standard cognitive assessment tests are selected from the Montreal Cognitive Assessment (MoCA), the Total Functional Capacity (TFC), Clinical Global Impression of Change (CGIC), and the Clinical Global Impression of Severity (CGIS). In certain embodiments, the one or more standard FUNCTIONAL assessment test is the TFC and the TFC score for the subject is in the range of 7-12, 8-12 or 8-11. In other embodiments, the one or more standard cognitive assessment test is the MoCA and the cognitive impairment assessment score for the subject is in the range of from 10-25, such as 10-21, 11-25, 19-25, or 11-21.In other embodiments, the one or more standard cognitive assessment test is the Mini Mental State Examination (MMSE) and a score less than or equal to 24 demotes some degree of cognitive impairment, such as a score of 17-24. In certain embodiments, the method is applied to subjects having, suspected of having, or diagnosed with Alzheimer's disease, Parkinson's disease, Huntington's disease, Down syndrome, ataxia, amyotrophic lateral sclerosis (ALS), frontotemporal dementia (FTD), HIV-related cognitive impairment, CNS Lupus, mild cognitive impairment, mild dementia, moderate cognitive impairment or a combination thereof. In other embodiments, the method is applied to subjects having, suspected of having, or diagnosed with Alzheimer's disease or Huntington's disease.

In the methods of this aspect of the disclosure, the antibody or antigen-binding fragment thereof that specifically binds to SEMA4D may comprise a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively or comprises a variable heavy chain (VH) comprising VHCDRs 1-3 comprising SEQ ID NOs 42, 43, and 44, respectively, and a variable light chain (VL) comprising VLCDRs 1-3 comprising SEQ ID NOs 46, 47, and 48, respectively. In other embodiments, the antibody or antigen binding fragment thereof inhibits SEMA4D binding to its receptor. In further embodiments, the receptor is selected from Plexin-B1 and Plexin-B2 and in certain embodiments, the antibody or antigen binding fragment thereof inhibits SEMA4D-mediated signal transduction.

Another aspect of the disclosure provides methods for predicting whether a semaphorin-4D (SEMA4D) antagonist antibody or antigen-binding fragment thereof will be effective in treating a subject having, determined to have, or suspected of having a neurodegenerative disorder. This method comprises (i) determining the cognitive and/or functional impairment assessment score for the subject in one or more standard cognitive and/or functional assessment tests; and (ii) predicting a positive response to treatment if the score satisfies a predetermined value indicative of mild cognitive impairment (MCI), mild dementia, moderate cognitive impairment, or Stage I or Stage II Huntington's disease. In certain embodiments, the one or more standard cognitive assessment tests are selected from the Montreal Cognitive Assessment (MoCA), the Total Functional Capacity (TFC), Clinical Global Impression of Change (CGIC), and the Clinical Global Impression of Severity (CGIS). In certain other embodiments, the one or more standard cognitive assessment test is the TFC and the cognitive impairment assessment score for the subject is in the range of 7-12, 8-12 or 8-11. In other embodiments, the one or more standard cognitive assessment test is the MoCA and the cognitive impairment assessment score for the subject is in the range of from 10-25, such as 10-21, 11-25, 19-25, or 11-21. In certain embodiments, the method is applied to subjects having, suspected of having, or diagnosed with Alzheimer's disease, Parkinson's disease, Huntington's disease, Down syndrome, ataxia, amyotrophic lateral sclerosis (ALS), frontotemporal dementia (FTD), HIV-related cognitive impairment, CNS Lupus, mild cognitive impairment, mild dementia, moderate cognitive impairment or a combination thereof. In other embodiments, the method is applied to subjects having, suspected of having, or diagnosed with Alzheimer's disease or Huntington's disease.

In the methods of this aspect of the disclosure, the antibody or antigen-binding fragment thereof that specifically binds to SEMA4D may comprise a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively or comprises a variable heavy chain (VH) comprising VHCDRs 1-3 comprising SEQ ID NOs 42, 43, and 44, respectively, and a variable light chain (VL) comprising VLCDRs 1-3 comprising SEQ ID NOs 46, 47, and 48, respectively. In other embodiments, the antibody or antigen binding fragment thereof inhibits SEMA4D binding to its receptor. In further embodiments, the receptor is selected from Plexin-B1 and Plexin-B2 and in certain embodiments, the antibody or antigen binding fragment thereof inhibits SEMA4D-mediated signal transduction.

In another aspect of the disclosure, methods for using semaphorin 4D binding molecules to alleviate symptoms in a subject having, determined to have, or suspected of having a neurodegenerative disorder are provided. According to this aspect of the disclosure illustrated herein, there is provided a method for treating a subject having, determined to have, or suspected of having a neurodegenerative disorder including determining (i) determining the cognitive and/or functional impairment assessment score for the subject in one or more standard cognitive and/or functional assessment tests; and (ii) administering a therapeutically effective amount of an isolated antibody or antigen-binding fragment thereof that specifically binds to semaphorin-4D (SEMA4D) if the score satisfies a predetermined value indicative of mild cognitive impairment (MCI), mild dementia, moderate cognitive impairment, or Stage I or Stage II Huntington's disease. In certain embodiments of this aspect of the disclosure, the one or more standard cognitive assessment tests are selected from the Montreal Cognitive Assessment (MoCA), the Total Functional Capacity (TFC), the Clinical Global Impression of Change (CGIC) test, and the Clinical Global Impression of Severity (CGIS) test. In other embodiments, the one or more standard cognitive assessment test is the TFC and the cognitive impairment assessment score for the subject is in the range of 7-12, such as 7-11, 8-12 or 8-11. In yet other embodiments, the one or more standard cognitive assessment test is the MoCA and the cognitive impairment assessment score for the subject is in the range of from 10-25, such as 10-21, 11-25, 19-25, or 11-21. In certain embodiments, the method is applied to subjects having, suspected of having, or diagnosed with Alzheimer's disease, Parkinson's disease, Huntington's disease, Down syndrome, ataxia, amyotrophic lateral sclerosis (ALS), frontotemporal dementia (FTD), HIV-related cognitive impairment, CNS Lupus, mild cognitive impairment, mild dementia, moderate cognitive impairment or a combination thereof. In other embodiments, the method is applied to subjects having, suspected of having, or diagnosed with Alzheimer's disease or Huntington's disease.

In the methods of this aspect of the disclosure, the antibody or antigen-binding fragment thereof that specifically binds to SEMA4D may comprise a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively or comprises a variable heavy chain (VH) comprising VHCDRs 1-3 comprising SEQ ID NOs 42, 43, and 44, respectively, and a variable light chain (VL) comprising VLCDRs 1-3 comprising SEQ ID NOs 46, 47, and 48, respectively. In other embodiments, the antibody or antigen binding fragment thereof inhibits SEMA4D binding to its receptor. In further embodiments, the receptor is selected from Plexin-B1 and Plexin-B2 and in certain embodiments, the antibody or antigen binding fragment thereof inhibits SEMA4D-mediated signal transduction.

In further embodiments of this aspect of the disclosure, the treatment results in a decrease, reduction, slowing or stopping of the incidence of symptoms associated with the neurodegenerative disorder; a decrease, reduction or lessening of the severity of symptoms associated with the neurodegenerative disorder; or improves the quality of life of the subject. In certain embodiments, the symptoms are selected from neuropsychiatric symptoms, cognitive symptoms, motor dysfunction and any combination thereof.

In another aspect of the disclosure, there is provided an isolated antibody or antigen-binding fragment thereof which specifically binds to semaphorin-4D (SEMA4D) for use in a method of treating a subject that has, is suspected of having or is diagnosed with a neurodegenerative disorder, wherein the subject is assessed to have a cognitive and/or functional impairment assessment score in one or more standard cognitive and/or functional assessment tests that is indicative of mild cognitive impairment, mild dementia or moderate dementia, or Stage I or Stage II Huntington's disease. In certain embodiments of this aspect, the antibody or antigen-binding fragment thereof that specifically binds to SEMA4D may comprise a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively or comprises a variable heavy chain (VH) comprising VHCDRs 1-3 comprising SEQ ID NOs 42, 43, and 44, respectively, and a variable light chain (VL) comprising VLCDRs 1-3 comprising SEQ ID NOs 46, 47, and 48, respectively. In other embodiments, the antibody or antigen binding fragment thereof inhibits SEMA4D binding to its receptor. In further embodiments, the receptor is selected from Plexin-B1 and Plexin-B2 and in certain embodiments, the antibody or antigen binding fragment thereof inhibits SEMA4D-mediated signal transduction. The isolated antibodies of this aspect may be used in any of the methods of the disclosure.

### BRIEF DESCRIPTION OF THE FIGURES

FIG. 1: shows categorical changes in the Clinical Global Impression of Change (CGIC) of early manifest patients treated with placebo (PBO) or an anti-SEMA4D antibody (Pepinemab; PEPI) during the primary analysis period from baseline to Months 3, 5, 11, and 17 by subgroup with baseline Unified Huntington Disease Rating Scale (UHDRS) Total Functional Capacity score (HDRS-TFC) of 11 or 12-13. FIG. 1A shows the categorical change in CGIC of subjects with a baseline TFC score of 11. FIG. 1B shows the categorical change in CGIC of subjects with a baseline TFC score of 12 and 13.

FIG. 2: Huntington Disease Cognitive Assessment Battery (HD-CAB) composite test results. FIG. 2A shows the change (CHG) from baseline (mean+standard error of measurement (SEM)) of HD-CAB composite score in patients with early manifest HD having a baseline MoCA score <26 treated with placebo (PBO) or an anti-SEMA4D antibody (Pepinemab; PEPI B1) over a period of 18months. FIG. 2B shows the change (CHG) from baseline (mean+standard error of measurement (SEM)) of HD-CAB composite score in patients with early manifest HD having a baseline MoCA score of ≥26 treated with placebo or an anti-SEMA4D antibody (Pepinemab; PEPI) over a period of 18 months.

FIG. 3: Total Motor Score (TMS) stratified by MoCA. FIG. 3A shows the change (CHG) from baseline in the TMS (mean+standard error of measurement (SEM)) in patients with early manifest HD having a baseline MoCA score of <26 treated with placebo (PBO) or an anti-SEMA4D antibody (Pepinemab; PEPI)) over 18 months. FIG. 3B shows the change (CHG) from baseline (mean+standard error of measurement (SEM)) in patients with early manifest HD having a baseline MoCA score of ≥26treated with placebo or an anti-SEMA4D antibody (Pepinemab; PEPI) over 18 months.

### DETAILED DESCRIPTION OF THE DISCLOSURE

### Definitions

It is to be noted that the term “a” or “an” entity refers to one or more of that entity; for example, “an anti-SEMA4D antibody” is understood to represent one or more anti-SEMA4D antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the terms “neurodegenerative disorder” or “neurodegenerative disease” are used interchangeably and refer to a central nervous system (CNS) disorder that is characterized by the death of neurons in one or more regions of the nervous system and the subsequent functional impairment of the affected parties. Examples of neurodegenerative disorders include, without limitation, Alzheimer's disease, Parkinson's disease, Huntington's disease, Down syndrome, ataxia, amyotrophic lateral sclerosis (ALS), frontotemporal dementia (FTD), HIV-related cognitive impairment (HAND, HIV-Associated Neurocognitive Disorder), CNS Lupus, mild cognitive impairment, mild dementia, and moderate cognitive impairment. Neurodegenerative diseases have an enormous impact on the lives of affected individuals and their families as well as society as a whole.

As used herein, the term “Alzheimer's disease” (AD) refers to a progressive disease initially manifesting itself with partial amnesia, and later restlessness, disorientation, aphasia, agnosia or apraxia (cognitive decline), dementia and sometimes euphoria or depressions. The disease typically starts at 40 to 90 years of age and predominantly affects females. As to its prevalence, estimations are about 13% of the population above 65 years age.

As used herein, the term “Huntington's disease” (HD) refers to a neurodegenerative disease, which is due to expansion of a poly-glutamine tract at the N-terminus of the protein huntingtin (expressed by the HTT gene) where the expansion can be more than 35-40 repetitions of the amino acid glutamine in the mutated protein (mHTT). The disease presents with progressive neuronal death in different brain areas, including toxicity in medium-sized spiny neurons of the striatum that determines the appearance of the classic motor incoordination and movements such as “Chorea.” The mechanism of action of mHTT has been described as both gain and loss of function compared with the wild-type protein and involves the acquisition or loss of competence to interact with various proteins in different cellular compartments.

The term “Mild Cognitive Impairment” (MCI) as used herein refers to the stage between the expected cognitive decline of normal aging and the more serious decline of dementia. MCI is characterized by memory, language, thinking and/or judgment issues. For neurodegenerative diseases, MCI can be an early stage of the disease continuum including for Alzheimer's disease if the hallmark changes in the brain are present. Individuals with MCI who have an abnormal brain positron emission tomography (PET) scan or spinal fluid test for amyloid beta protein (one of the two hallmarks of Alzheimer's disease), are considered to have a diagnosis of MCI due to Alzheimer's disease. MCI is indicated by a MoCA score of from 18-25, a Qmci score of <62/100, for example, a MMSE score of 24-27/30, an ACE-III score of 80-90/100, a RUDAS score of 23-26/30. The skilled practitioner can identify scores from other CI tests that indicate MCI, such as test scores that correspond to the applicable MoCA score or TFC score, where applicable.

The term “Mild Dementia” as used herein refers to a state of moderate memory loss and disorientation associated with impaired problem solving. At this stage, afflicted individuals may be able to function independently but may have short-term memory lapses, and difficulties with complex tasks or problem solving. Mild dementia is identified by a MSSE: score of 8-23/30, an ACE-III score of 65-76/100, a MOCA score of 11-17/30, or a RUDAS score of 17-22/30, for example.

The term “Moderate Cognitive Impairment” or “Moderate Dementia” as used herein refers to a stage of dementia where the impaired individual exhibits clear, visible signs of mental impairment. While moderate cognitive impairment is considered mild or early stage dementia, the medical terminology for this stage of dementia is “moderate cognitive decline.” Moderate Cognitive Impairment is indicated by a MMSE score of 10-18/30, a MoCA score of 3-10/30, an ACE-III score of 35-64/100, or a RUDAS score or 10-16/30, for example. The skilled practitioner can identify scores from other CI tests that indicate moderate cognitive impairment, such as test scores that correspond to the applicable MoCA score or TFC score

The term “Severe Dementia” refers to a stage of dementia associated with profound impairment of cognition and function. Symptoms include severe memory impairment, disorientation, limited or lost spoken language skills, incontinence, lack of a capacity for making judgements, high dependency on others for personal care, etc. Severe dementia is indicated by a MMSE:<10/30, ACE-III: <35/100, MOCA: <6/30 (or not testable), or a RUDAS: <10/30 for example. The skilled practitioner can identify scores from other CI tests that indicate severe dementia, such as test scores that correspond to the applicable MoCA score or TFC score.

The term “therapeutically effective amount” refers to an amount of an antibody, polypeptide, polynucleotide, small organic molecule, or other drug effective to “treat” a disease or disorder in a subject. In the case of a neurodegenerative disorder, the therapeutically effective amount of the drug can alleviate symptoms of the disorder; decrease, reduce, retard or stop the incidence of symptoms; decrease, reduce, retard the severity of symptoms; inhibit, e.g., suppress, retard, prevent, stop, or reverse the manifestation of symptoms; relieve to some extent one or more of the symptoms associated with the disorder; reduce morbidity and mortality; improve quality of life; or a combination of such effects.

The term “symptoms” as referred to herein refers to, e.g., 1) neuropsychiatric symptoms, 2) cognitive symptoms, and 3) motor dysfunction. Examples of neuropsychiatric symptoms include, for instance, anxiety-like behavior. Examples of cognitive symptoms include, for instance, learning and memory deficits. Examples of motor dysfunction include, for instance, locomotion.

The term “Total Functional Capacity (TFC)” as referred to herein is in reference to the Shoulston-Fahn standardized scale used to assess capacity to work, handle finances, perform domestic chores and self-care tasks, and live independently. The TFC scale ranges from 13 (normal) to 0 (severe disability), with higher scores indicating higher functioning. The TFC has been partitioned into five stages that indicate levels of disease severity based on functional decline. TFC scores from 11-13 represent stage I (least severe); 7-10, stage II; 3-6, stage III; 1-2, stage IV; and score of 0 is stage V (most severe). TFC is the standard clinical assessment tool in determining the stage of Huntington's disease. (Unified Huntington's Disease Rating Scale: reliability and consistency. Huntington Study Group. Mov Disord. 1996;11(2):136-142). TFC and Total Motor Score (TMS) are components of the UHDRS.

As used herein, the term “Unified Huntington's Disease Rating Scale (UHDRS) Total Motor Score (TMS)” refers to the most widely used measure of motor function in HD. The UHDRS-TMS assesses motor features of HD with standardized ratings in the following five domains: eye movement, chorea (jerky movement), dystonia (muscle spasm and twisting), bradykinesia (slowness in movement), and rigidity (stiffness). 31 Items in each of the five domains are individually rated on a five-point scale ranging from 0 (normal) to 4 (most severe impairment). The sum of the scores of all 31 items is referred to as the UHDRS-TMS. The range of the UHDRS-TMS is 0 to 124, with higher scores indicating more severe motor impairment.

The term “Montreal Cognitive Assessment” (MoCA) as used herein refers to a validated rapid cognitive screening test designed to assist in detection of mild cognitive impairment (MCI) and stages of Alzheimer's disease (AD), as well as other neurodegenerative disorders. It assesses different cognitive domains: attention and concentration, executive functions, memory, language, visuo-constructional skills, conceptual thinking, calculations, and orientation. The total possible score is 30 points; a score of 26 or above is considered normal and scores 19-25 suggest mild cognitive impairment (MCI), scores of 11-21 are considered Mild AD, a score of 6-10 suggests moderate dementia. MoCA scores can be used to assess any disorder with associated dementia or cognitive decline. (Nasreddine ZS et al. The Montreal Cognitive Assessment, MoCA: a brief screening tool for mild cognitive impairment. J Am Geriatr Soc. 2005; 53 (4): 695-9).

The term “Huntington's Disease Cognitive Assessment Battery” (HD-CAB) as used herein refers to six tests which constitute the Huntington's Disease Cognitive Assessment Battery (HD-CAB): Symbol Digit Modalities Test (SDMT), Paced Tapping (PTAP), One Touch Stockings of Cambridge (OTS), Emotion Recognition (EMO), Trail Making B (TRAILS-B), and the Hopkins Verbal Learning Test (HVLT R). These tests are used to demonstrate sensitivity to disease status (Cohen's d effect sizes: early HD=−1.38 to −1.90 and pre-HD=−0.41 to −0.78), and acceptable reliability (r's 0.73-0.93). A composite score shows large effect sizes (early HD=−2.44 and pre-HD=−0.87) and high reliability (r=0.95). The battery has high sensitivity to disease status, with large effect sizes, and high reliability, and well-characterized psychometrics and practice effects. The battery of tests that comprise the HD-CAB offers sufficient range for detecting both symptomatic improvement and slowing of decline. The HD-CAB composite, the statistic summarizing the results of all six HD-CAB component tests, is the sum of the z-scores of all six tests. The HD-CAB measures the following cognitive domains: executive function, attention, memory, visuospatial processing, timing, and emotion processing. In multifaceted diseases like HD, the HD-CAB composite is more likely than a single HD-CAB component to capture the complexity of disease. Depending on the individual and where people are in the progression of disease, certain cognitive domains are affected more than others. A single HD-CAB component measuring a single domain is unlikely to capture the degree and range of cognitive impairment of HD in all individuals. Combining measures into a composite endpoint that reflects multiple cognitive domains has the ability to capture cognitive effects more completely. A higher value on the HD-CAB composite indicates higher cognitive functioning. (Stout J C, Queller S, Baker K N, et al. HD-CAB: a cognitive assessment battery for clinical trials in Huntington's disease. Mov Disord. 2014;29(10): 1281-1288).

The Clinical Global Impressions scale (CGI) initially consists of three different global measures: Severity of illness: (CGI-S); Global Improvement (CGI-I); Efficacy index (CGI-E). The CGI-I score generally tracks with the CGI-S such that improvement in one follows the other. CGI-S and CGI-I scores can occasionally be dissociated. (Busner and Targum, The Clinical Global Impressions Scales. Psychiatry (Edgemont). (2007) 4:28-37, incorporated herein)

The term “Clinical Global Impression of Change (or Improvement)” (CGIC) refers to a scale that requires the clinician to assess how much the patient's illness has improved or worsened relative to a baseline state at the beginning of a treatment intervention. The CGIC is a single item question about the individual's overall disease status. It assesses overall response to study drug through a 7-point Likert scale, ranging from very much worse (−3) to very much improved (+3). Treatment response ratings should take account of both therapeutic efficacy and treatment-related adverse events. Each component of the CGIC is rated separately. (Guy, W. (1976) Assessment Manual for Psychopharmacology. Rockville, Md.: US Department of Health, Education, and Welfare Public Health Service Alcohol, Drug Abuse, and Mental Health Administration).

The term “Clinical Global Impression-Severity scale (CGI-S)” as used herein refers to a seven-point scale that requires the clinician to rate the severity of the patient's illness at the time of assessment, relative to the clinician's past experience with patients who have the same diagnosis. Considering total clinical experience, a patient is assessed on severity of mental illness at the time of rating. Scores range from 1 (Normal) to 7 (extremely mentally ill).

CGIC and MoCA are utilized in a variety of neurodegenerative diseases, including but not limited to AD; TFC, TMS, and HD-CAB are specific to HD.

The term “Mini Mental State Examination” as used herein refers to a standardized tool that can be used to systematically and thoroughly assess mental status. It is an 11-question measure that tests five areas of cognitive function: orientation, registration (immediate memory), attention and calculation, recall (short-term memory), and language. The maximum score is 30. A score of 23 or lower is indicative of cognitive impairment mild cognitive impairment: 24-27/30; mild dementia: 18-23/30; moderate dementia: 10-18/23). Scores of 28-30 out of 30 are considered normal; the National Institute for Health and Care Excellence (NICE) classifies 21-24 as mild, 10-20 as moderate dementia and <10 as severe impairment.

The term “Mini-Cog™” as used herein refers to a screening tool used to detect cognitive impairment quickly during both routine visits and hospitalizations. The Mini-Cog™ serves as an effective triage tool to identify individuals in need of more thorough evaluation. The Clock Drawing Test (CDT) component of the Mini-Cog™ allows clinicians to quickly assess numerous cognitive domains including cognitive function, memory, language comprehension, visual-motor skills, and executive function and provides a visible record of both normal and impaired performance that can be tracked over time. The Mini-Cog™ is often used in combination with other tests to detect cognitive impairment.

The “Quick Mild Cognitive Impairment screen” (Qmci) as referenced herein is a short screening test for cognitive impairment that was developed as a rapid, valid, and reliable instrument for the early detection and differential diagnosis of MCI and dementia (O'Caoimh R, Gao Y, McGlade C, Healy L, Gallagher P, Timmons S, Molloy D W (2012) Comparison of the Quick Mild Cognitive Impairment (Qmci) screen and the MMSE in screening for mild cognitive impairment. Age Ageing 41, 624-629.; O'Caoimh R, Gao Y, Gallagher P, Eustace J, McGlade C, Molloy D W (2013) Which part of the Quick mild cognitive impairment screen (Qmci) discriminates between normal cognition, mild cognitive impairment and dementia?, Age Ageing 42, 324-330). The Qmci correlates with the standardized Alzheimer's Disease Assessment Scale-cognitive section, Clinical Dementia Rating scale and the Lawton-Brody activities of daily living scale. (O'Caoimh R, Svendrovski A, Johnston B, Gao Y, McGlade C, Timmons S , Eustace J, Guyatt G, Molloy D W (2014) Comparison of the Quick mild cognitive impairment screen (Qmci) to the Standardized Alzheimer's Disease Assessment Scale-cognitive section (SADAS-cog) in clinical trials. J Clin Epidemiol 67, 87-92). Both the Qmci and MoCA have excellent accuracy in separating MCI from dementia, and normal cognition and subjective memory complaints (SMC) from MCI and cognitive impairment (CI). Qmci has excellent sensitivity and specificity. At a scoring cut-off of <62/100, Cmci has been shown to have 90% sensitivity and 87% specificity for detecting cognitive impairment. (O'Caoimh R, Gao Y, Gallagher P, Eustace J, Molloy W (2014).

As used herein, the term “Alzheimer's Disease Assessment Scale-Cognitive Subscale” (ADAS-Cog Test) is in reference to one of the most frequently used tests to measure cognition in research studies and clinical trials for new drugs and other interventions. The test primarily measures language and memory. The ADAS-Cog was developed as a two-part scale: one that measured cognitive functions and one that measured non-cognitive functions such as mood and behavior. Most current research uses the ADAS-Cog Subscale, which is the subscale that measures cognitive ability. (Kueper J K, Speechley M, Montero-odasso M. The Alzheimer's Disease Assessment Scale-Cognitive Subscale (ADAS-Cog): modifications and responsiveness in pre-dementia populations. A narrative review. J Alzheimer's Dis. 2018;63(2):423-444. doi:10.3233/JAD-170991). The ADAS-Cog helps evaluate cognition and differentiates between normal cognitive functioning and impaired cognitive functioning. It is especially useful for determining the extent of cognitive decline and can help evaluate which stage of Alzheimer's disease a person is in, based on his answers and score. The ADAS-Cog is often used in clinical trials because it can determine incremental improvements or declines in cognitive functioning. (Cano S J, Posner H B, Moline M L, et al. The ADAS-cog in Alzheimer's disease clinical trials: psychometric evaluation of the sum and its parts. Journal of Neurology, Neurosurgery & Psychiatry 2010;81:1363-1368) Scoring on the ADAS-Cog test is based on points earned for errors in each task of the ADAS-Cog for a total score ranging from 0 to 70. The greater the dysfunction, the greater the score. A score of 70 represents the most severe impairment and 0 represents the least impairment. (Food and Drug Association. ADAS-Cog Administration and Scoring Manual).

As referred to herein the “Clinical Dementia Rating Scale Sum of Boxes” (CDR-SOB) is a test used to stage the severity of AD. A Texas Alzheimer's Research Consortium Study (Sid E. O'Bryant, PhD, Stephen C. Waring, DVM, PhD, C. Munro Cullum, PhD, James Hall, PhD, Laura Lacritz, PhD, Paul J. Massman, PhD, Philip J. Lupo, M P H, Joan S. Reisch, PhD, Rachelle Doody, MD, PhD, and Texas Alzheimer's Research Consortium, Arch Neurol. 2008;65(8): 1091-1095. doi:10.1001/archneur.65.8.1091) concluded that mild AD spans CDR-SOB scores from 4.5 to 9.0, and moderate AD spans CDR-SOB scores from 9.5 to 15.5.

As referred to herein, “Addenbrooke's Cognitive Examination III” (ACE-III) is a screening test that is composed of tests of attention, orientation, memory, language, visual perceptual and visuospatial skills. It is useful in the detection of cognitive impairment, especially in the detection of Alzheimer's disease and fronto-temporal dementia. The ACE-III test consists of 19 activities which test five cognitive domains: attention, memory, fluency, language and visuospatial processing. The results of each activity are scored to give a total score out of 100 (18 points for attention, 26 for memory, 14 for fluency, 26 for language, 16 for visuospatial processing). The score needs to be interpreted in the context of the patient's overall history and examination, but a score of 88 and above is considered normal; below 83 is indicative of dementia.

The “Rowland Universal Dementia Assessment Scale” (RUDAS) as referred to herein is a short cognitive screening instrument designed to minimize the effects of cultural learning and language diversity on the assessment of baseline cognitive performance. The RUDAS seems likely to have less cultural and educational bias so it is easily interpreted by an interpreter during an assessment. The RUDAS consists of a series of questions aimed at assessing memory, visuospatial orientation, praxis, visuoconstructional drawing, memory recall and language. A score of 23-30 is considered normal; lower scores indicate greater impairment.

The term “determining the cognitive or functional impairment assessment score,” is used herein to mean that testing of the cognitive ability and/or functional ability of a subject is carried out using a standardized screening test for MCI/dementia (cognitive ability) or HD (functional ability), e.g., the MoCA test or the TFC test, respectively, to assess cognitive impairment (CI) such as CI associated with neurodegenerative disorders and/or functional impairment or functional deficit associated with HD, for example. The results of any of the CI or functional tests described herein, such as the MoCA or TFC tests, are applied to the appropriate scale to obtain an overall score for that subject. Similarly, the terms “determining the Montreal Cognitive Assessment (MoCA) score” or “determining the Total Functional Capacity (TFC) score” means testing of the cognitive ability or total functional ability of a subject using MoCA test or TFC test, respectively.

Testing is generally done by a qualified and/or certified healthcare provider. It is not necessary and is often not the case that the individual(s) who carries out testing and/or scoring also treats the subject. For the purposes of the methods of the disclosure, test scores such as the MoCA or TFC scores for example can be obtained from a person, such as a qualified and/or certified healthcare provider for assessing whether to administer treatment.

Terms such as “treating” or “treatment” or “to treat” or “alleviating” or “to alleviate” or “improving” or “to improve” refer to both 1) therapeutic measures that cure, slow down, lessen symptoms of, reverse, and/or halt progression of a diagnosed pathologic condition or disorder and 2) prophylactic or preventative measures that prevent and/or slow the development of a targeted pathologic condition or disorder. Thus those in need of treatment include those already with the disorder; those prone to have the disorder; and those in whom the disorder is to be prevented. Beneficial or desired clinical results include, but are not limited to, alleviation of symptoms, diminishment of extent of disease, stabilization (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “patient” is meant any subject, particularly a human.

As used herein, the phrase “a subject that would benefit from administration of an anti-SEMA4D antibody” includes human subjects, that would benefit from administration of an anti-SEMA4D antibody or other SEMA4D binding molecule used, e.g., for detection of a SEMA4D polypeptide (e.g., for a diagnostic procedure) and/or from treatment, i.e., palliation or prevention of a disease, with an anti-SEMA4D antibody or other SEMA4D binding molecule.

A “binding molecule” or “antigen binding molecule” of the present disclosure refers in its broadest sense to a molecule that specifically binds an antigenic determinant. In one embodiment, the binding molecule specifically binds to SEMA4D, e.g., to a transmembrane SEMA4D polypeptide of about 150 kDa or a soluble SEMA4D polypeptide of about 120 kDa (commonly referred to as sSEMA4D). In some embodiments, a binding molecule of the disclosure is an antibody or an antigen binding fragment thereof. In another embodiment, a binding molecule of the disclosure comprises at least one heavy or light chain CDR of an antibody molecule. In another embodiment, a binding molecule of the disclosure comprises at least two CDRs from one or more antibody molecules. In another embodiment, a binding molecule of the disclosure comprises at least three CDRs from one or more antibody molecules. In another embodiment, a binding molecule of the disclosure comprises at least four CDRs from one or more antibody molecules. In another embodiment, a binding molecule of the disclosure comprises at least five CDRs from one or more antibody molecules. In another embodiment, a binding molecule of the disclosure comprises at least six CDRs from one or more antibody molecules.

The present disclosure is directed to methods of selecting subjects for treatment with an anti-SEMA4 antibody , methods of determining whether a semaphorin 4D (SEMA4D) antagonist antibody or antigen-binding fragment thereof will be effective in treating and methods of treating or alleviating symptoms in subjects having, suspected of having or diagnosed with a neurodegenerative disorder. The methods of the disclosure comprise determining the subject's cognitive impairment assessment score through use of any one or more of the standardized cognitive assessment tests including those tests described herein or other valid standardized cognitive assessment tests, such as the MoCA and/or TFC for example (as described herein) and selecting the subject for treatment, determining whether treatment is likely to be successful, or administering to the subject an anti-SEMA4D binding molecule, e.g., an antibody, or antigen-binding fragment, variant, or derivative thereof (i.e., treating), as applicable, if the test score, e.g., MoCA and/or TFC scores satisfy a predetermined value(s). Unless specifically referring to full-sized antibodies such as naturally occurring antibodies, the term “anti-SEMA4D antibody” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

As used herein, “human” or “fully human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example, in U.S. Pat. No. 5,939,598 by Kucherlapati et al. “Human” or “fully human” antibodies also include antibodies comprising at least the variable domain of a heavy chain, or at least the variable domains of a heavy chain and a light chain, where the variable domain(s) have the amino acid sequence of human immunoglobulin variable domain(s).

“Human” or “fully human” antibodies also include “human” or “fully human” antibodies, as described above, that comprise, consist essentially of, or consist of, variants (including derivatives) of antibody molecules (e.g., the VH regions and/or VL regions) described herein, which antibodies or fragments thereof immunospecifically bind to a SEMA4D polypeptide or fragment or variant thereof. Standard techniques known to those of skill in the art can be used to introduce mutations in the nucleotide sequence encoding a human anti-SEMA4D antibody, including, but not limited to, site-directed mutagenesis and PCR-mediated mutagenesis which result in amino acid substitutions. In some embodiments, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3.

In certain embodiments, the amino acid substitutions are conservative amino acid substitutions, discussed further below. Alternatively, mutations can be introduced randomly along all or part of the coding sequence, such as by saturation mutagenesis, and the resultant mutants can be screened for biological activity to identify mutants that retain activity (e.g., the ability to bind a SEMA4D polypeptide, e.g., human, murine, or both human and murine SEMA4D). Such variants (or derivatives thereof) of “human” or “fully human” antibodies can also be referred to as human or fully human antibodies that are “optimized” or “optimized for antigen binding” and include antibodies that have improved affinity to antigen.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al. (1988) Antibodies: A Laboratory Manual (2nd ed.; Cold Spring Harbor Laboratory Press).

As used herein, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 γ4. γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, IgA2, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ) Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL or VK) and heavy (VH) chain portions determine antigen recognition and specificity. Conversely, the constant domains of the light chain (CL) and the heavy chain (typically CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains typically comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs) within these variable domains, of an antibody combine to form the variable region that defines a three dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule can consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable domain by one of ordinary skill in the art, since they have been precisely defined (see below).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al. (1983) U.S. Dept. of Health and Human Services, “Sequences of Proteins of Immunological Interest” and by Chothia and Lesk, J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers that encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al. (1983) U.S. Dept. of Health and Human Services, “Sequence of Proteins of Immunological Interest.” Unless otherwise specified, references to the numbering of specific amino acid residue positions in an anti-SEMA4D antibody or antigen-binding fragment, variant, or derivative thereof of the present disclosure are according to the Kabat numbering system.

Antibodies or antigen-binding fragments, variants, or derivatives thereof of the disclosure include, but are not limited to, polyclonal, monoclonal, multispecific and bispecific in which at least one arm is specific for SEMA4D, human, humanized, primatized, or chimeric antibodies, single-chain antibodies, epitope-binding fragments, e.g., Fab, Fab′ and F(ab′)₂, Fd, Fvs, single-chain Fvs (scFv), disulfide-linked Fvs (sdFv), fragments comprising either a VL or VH domain, fragments produced by a Fab expression library, and anti-idiotypic (anti-Id) antibodies (including, e.g., anti-Id antibodies to anti-SEMA4D antibodies disclosed herein). ScFv molecules are known in the art and are described, e.g., in U.S. Pat. No. 5,892,019. Immunoglobulin or antibody molecules of the disclosure can be of any type (e.g., IgG, IgE, IgM, IgD, IgA, and IgY), class (e.g., IgG1, IgG2, IgG3, IgG4, IgA1, and IgA2, etc.), or subclass of immunoglobulin molecule.

As used herein, the term “heavy chain portion” includes amino acid sequences derived from an immunoglobulin heavy chain. In certain embodiments, a polypeptide comprising a heavy chain portion comprises at least one of: a VH domain, a CH1 domain, a hinge (e.g., upper, middle, and/or lower hinge region) domain, a CH2 domain, a CH3 domain, or a variant or fragment thereof. For example, a binding polypeptide for use in the disclosure can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, a binding polypeptide for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

In certain anti-SEMA4D antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the disclosure are not identical. For example, each monomer can comprise a different target binding site, forming, for example, a bispecific antibody.

The heavy chain portions of a binding molecule for use in the methods disclosed herein can be derived from different immunoglobulin molecules. For example, a heavy chain portion of a polypeptide can comprise a Cm domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain, e.g., a kappa or lambda light chain. In some aspects, the light chain portion comprises at least one of a VL or CL domain.

Anti-SEMA4D antibodies, or antigen-binding fragments, variants, or derivatives thereof disclosed herein can be described or specified in terms of the epitope(s) or portion(s) of an antigen, e.g., a target polypeptide disclosed herein (e.g., SEMA4D) that they recognize or specifically bind. The portion of a target polypeptide that specifically interacts with the antigen binding domain of an antibody is an “epitope,” or an “antigenic determinant.” A target polypeptide can comprise a single epitope, but typically comprises at least two epitopes, and can include any number of epitopes, depending on the size, conformation, and type of antigen. Furthermore, it should be noted that an “epitope” on a target polypeptide can be or can include non-polypeptide elements, e.g., an epitope can include a carbohydrate side chain.

The minimum size of a peptide or polypeptide epitope for an antibody is thought to be about four to five amino acids. Peptide or polypeptide epitopes can contain, e.g., at least seven, at least nine or between at least about 15 to about 30 amino acids. Since a CDR can recognize an antigenic peptide or polypeptide in its tertiary form, the amino acids comprising an epitope need not be contiguous, and in some cases, can be on separate peptide chains. A peptide or polypeptide epitope recognized by anti-SEMA4D antibodies of the present disclosure can contain a sequence of at least 4, at least 5, at least 6, at least 7, at least 8, at least 9, at least 10, at least 15, at least 20, at least 25, or between about 15 to about 30 contiguous or non-contiguous amino acids of SEMA4D.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” can be deemed to have a higher specificity for a given epitope than antibody “B,” or antibody “A” can be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody that “preferentially binds” to a given epitope would more likely bind to that epitope than to a related epitope, even though such an antibody can cross-react with the related epitope.

By way of non-limiting example, an antibody can be considered to bind a first epitope preferentially if it binds the first epitope with a dissociation constant (K_{D}) that is less than the antibody's K_{D} for the second epitope. In another non-limiting example, an antibody can be considered to bind a first antigen preferentially if it binds the first epitope with an affinity that is at least one order of magnitude less than the antibody's K_{D} for the second epitope. In another non-limiting example, an antibody can be considered to bind a first epitope preferentially if it binds the first epitope with an affinity that is at least two orders of magnitude less than the antibody's K_{D} for the second epitope.

In another non-limiting example, an antibody can be considered to bind a first epitope preferentially if it binds the first epitope with an off rate (k(off)) that is less than the antibody's k(off) for the second epitope. In another non-limiting example, an antibody can be considered to bind a first epitope preferentially if it binds the first epitope with an affinity that is at least one order of magnitude less than the antibody's k(off) for the second epitope. In another non-limiting example, an antibody can be considered to bind a first epitope preferentially if it binds the first epitope with an affinity that is at least two orders of magnitude less than the antibody's k(off) for the second epitope. An antibody or antigen-binding fragment, variant, or derivative disclosed herein can be said to bind a target polypeptide disclosed herein (e.g., SEMA4D, e.g., human, murine, or both human and murine SEMA4D) or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. In certain aspects, an antibody of the disclosure can be said to bind a target polypeptide disclosed herein (e.g., SEMA4D, e.g., human, murine, or both human and murine SEMA4D) or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

An antibody or antigen-binding fragment, variant, or derivative disclosed herein can be said to bind a target polypeptide disclosed herein (e.g., SEMA4D, e.g., human, murine, or both human and murine SEMA4D) or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. In some embodiments, an antibody of the disclosure cab be said to bind a target polypeptide disclosed herein (e.g., SEMA4D, e.g., human, murine, or both human and murine SEMA4D) or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

An antibody is said to competitively inhibit binding of a reference antibody to a given epitope if it preferentially binds to that epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al. (1988) Antibodies: A Laboratory Manual (Cold Spring Harbor Laboratory Press, 2nd ed.) pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Anti-SEMA4D antibodies or antigen-binding fragments, variants, or derivatives thereof of the disclosure can also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, can actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody can be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody can be deemed “highly specific” for a certain epitope, if it does not bind any other analog, ortholog, or homolog of that epitope.

Anti-SEMA4D binding molecules, e.g., antibodies or antigen-binding fragments, variants or derivatives thereof, of the disclosure can also be described or specified in terms of their binding affinity to a polypeptide of the disclosure, e.g., SEMA4D, e.g., human, murine, or both human and murine SEMA4D. In certain aspects, the binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In certain embodiments, the anti-SEMA4D binding molecule, e.g., an antibody or antigen binding fragment thereof, of the disclosure binds human SEMA4D with a Kd of about 5×10⁻⁹ to about 6×10⁻⁹. In another embodiment, the anti-SEMA4D binding molecule, e.g., an antibody or antigen binding fragment thereof, of the disclosure binds murine SEMA4D with a Kd of about 1×10⁻⁹ to about 2×10⁻⁹.

As used herein, the term “chimeric antibody” will be held to mean any antibody wherein the immunoreactive region or site is obtained or derived from a first species and the constant region (which can be intact, partial or modified in accordance with the instant disclosure) is obtained from a second species. In certain embodiments the target binding region or site will be from a non-human source (e.g., mouse or primate) and the constant region is human.

As used herein, the term “engineered antibody” refers to an antibody in which the variable domain in either the heavy or light chain or both is altered by at least partial replacement of one or more CDRs from an antibody of known specificity and, if necessary, by partial framework region replacement and sequence changing. Although the CDRs can be derived from an antibody of the same class or even subclass as the antibody from which the framework regions are derived, it is envisaged that the CDRs will be derived from an antibody of different class, or from an antibody from a different species. An engineered antibody in which one or more “donor” CDRs from a non-human antibody of known specificity is grafted into a human heavy or light chain framework region is referred to herein as a “humanized antibody.” It is not always necessary to replace all of the CDRs with the complete CDRs from the donor variable domain to transfer the antigen binding capacity of one variable domain to another. Rather, one can transfer just those residues needed to maintain the activity of the target binding site need be transferred.

It is further recognized that the framework regions within the variable domain in a heavy or light chain, or both, of a humanized antibody can comprise solely residues of human origin, in which case these framework regions of the humanized antibody are referred to as “fully human framework regions” (for example, MAbs 1515/2503 or 67, disclosed in U.S. Patent Appl. Publication No. US 2010/0285036 A1 as MAb 2503, incorporated herein by reference in its entirety). Alternatively, one or more residues of the framework region(s) of the donor variable domain can be engineered within the corresponding position of the human framework region(s) of a variable domain in a heavy or light chain, or both, of a humanized antibody if necessary to maintain proper binding or to enhance binding to the SEMA4D antigen. A human framework region that has been engineered in this manner would thus comprise a mixture of human and donor framework residues, and is referred to herein as a “partially human framework region.”

For example, humanization of an anti-SEMA4D antibody can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent or mutant rodent CDRs or CDR sequences for the corresponding sequences of a human anti-SEMA4D antibody. See also U.S. Pat. Nos. 5,225,539; 5,585,089; 5,693,761; 5,693,762; 5,859,205; herein incorporated by reference. The resulting humanized anti-SEMA4D antibody would comprise at least one rodent or mutant rodent CDR within the fully human framework regions of the variable domain of the heavy and/or light chain of the humanized antibody. In some instances, residues within the framework regions of one or more variable domains of the humanized anti-SEMA4D antibody are replaced by corresponding non-human (for example, rodent) residues (see, for example, U.S. Pat. Nos. 5,585,089; 5,693,761; 5,693,762; and 6,180,370), in which case the resulting humanized anti-SEMA4D antibody would comprise partially human framework regions within the variable domain of the heavy and/or light chain.

Furthermore, humanized antibodies can comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the CDRs correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331:522-525 (1986); Riechmann et al., Nature 332:323-329 (1988); and Presta, Curr. Op. Struct. Biol. 2:593-596 (1992); herein incorporated by reference. Accordingly, such “humanized” antibodies can include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

As used herein, the term “healthcare provider” refers to individuals or institutions that directly interact and administer to living subjects, e.g., human patients. Disease assessment, testing and treatment are generally administered by healthcare providers. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

### II. Target Polypeptide Description

As used herein, the terms “Semaphorin 4D,” “SEMA4D” and “SEMA4D polypeptide” are used interchangeably, as are “SEMA4D” and “Sema4D.” In certain embodiments, SEMA4D is expressed on the surface of or secreted by a cell. In another embodiment, SEMA4D is membrane bound. In another embodiments, SEMA4D is soluble, e.g., sSEMA4D. In other embodiments, SEMA4D can include a full-sized SEMA4D or a fragment thereof, or a SEMA4D variant polypeptide, wherein the fragment of SEMA4D or SEMA4D variant polypeptide retains some or all functional properties of the full-sized SEMA4D.

The full-sized human SEMA4D protein is a homodimeric transmembrane protein consisting of two polypeptide chains of 150 kDa. SEMA4D belongs to the semaphorin family of cell surface receptors and is also referred to as CD100. Both human and mouse SEMA4D/Sema4D are proteolytically cleaved from their transmembrane form to generate 120-kDa soluble forms, indicating the existence of two Sema4D isoforms (Kumanogoh et al., J. Cell Science 116(7):3464 (2003)). Semaphorins consist of soluble and membrane-bound proteins that were originally defined as axonal-guidance factors during development which play an important role in establishing precise connections between neurons and their appropriate target. Structurally considered a class IV semaphorin, SEMA4D consists of an amino-terminal signal sequence followed by a characteristic ‘Sema’ domain, which contains 17 conserved cysteine residues, an Ig-like domain, a lysine-rich stretch, a hydrophobic transmembrane region, and a cytoplasmic tail.

A polypeptide chain of SEMA4D can include a signal sequence of about 13 amino acids and further includes a semaphorin domain of about 512 amino acids, an immunoglobulin-like (Ig-like) domain of about 65 amino acids, a lysine-rich stretch of 104 amino acids, a hydrophobic transmembrane region of about 19 amino acids, and a cytoplasmic tail of 110 amino acids. A consensus site for tyrosine phosphorylation in the cytoplasmic tail supports the predicted association of SEMA4D with a tyrosine kinase (Schlossman, et al., Eds. (1995) Leucocyte Typing V (Oxford University Press, Oxford).

SEMA4D is known to have at least three functional receptors, Plexin-B1,

Plexin-B2 and CD72. One of the receptors, Plexin-B1, is expressed in non-lymphoid tissues and has been shown to be a high affinity (1 nM) receptor for SEMA4D (Tamagnone et al., Cell 99:71-80 (1999)). SEMA4D stimulation of Plexin-B1 signaling has been shown to induce growth cone collapse of neurons, and to induce process extension collapse and apoptosis of oligodendrocytes (Giraudon et al., J. Immunol. 172:1246-1255 (2004); Giraudon et al., NeuroMolecular Med. 7:207-216 (2005)). After binding to SEMA4D, Plexin-B1 signaling mediates the inactivation of R-Ras, leading to a decrease in the integrin mediated attachment to the extracellular matrix, as well as to activation of RhoA, leading to reorganization of the cytoskeleton and cell migration. See Kruger et al. , Nature Rev. Mol. Cell Biol. 6:789-800 (2005); Pasterkamp, TRENDS in Cell Biology 15:61-64 (2005)). Plexin-B2, on the other hand, has an intermediate affinity for SEMA4D and recent reports indicate that Plexin-B2 regulates migration of cortical neurons and proliferation and migration of neuroblasts in the adult subventricular zone (Azzarelli et al,. Nat Commun 2014 Feb 27, 5:3405, DOI: 10.1038/ncomms4405; and Saha et al., J. Neuroscience, 2012 November 21, 32(47):16892-16905).

In lymphoid tissues CD72 is utilized as a low affinity (300nM) SEMA4D receptor (Kumanogoh et al., Immunity/13:621-631 (2000)). B cells and APCs express CD72, and anti-CD72 antibodies have many of the same effects as sSEMA4D, such as enhancement of CD40-induced B cell responses and B cell shedding of CD23. CD72 is thought to act as a negative regulator of B cell responses by recruiting the tyrosine phosphatase SHP-1, which can associate with many inhibitory receptors. Interaction of SEMA4D with CD72 results in the dissociation of SHP-1, and the loss of this negative activation signal. SEMA4D has been reported to promote T cell stimulation and B cell aggregation and survival in vitro. The addition of SEMA4D-expressing cells or sSEMA4D enhances CD40-induced B cell proliferation and immunoglobulin production in vitro, and accelerates in vivo antibody responses (Ishida et al., Inter. Immunol. 15:1027-1034 (2003); Kumanogoh and H. Kukutani, Trends in Immunol. 22:670-676 (2001)). sSEMA4D enhances the CD40 induced maturation of dendritic cells (DCs), including up-regulation of costimulatory molecules and increased secretion of IL-12. In addition, sSEMA4D can inhibit immune cell migration, which can be reversed by addition of blocking anti-SEMA4D antibodies (Elhabazi et al., J. Immunol. 166:4341-4347 (2001); Delaire et al., J. Immunol. 166:4348-4354 (2001)).

Sema4D is expressed at high levels in lymphoid organs, including the spleen, thymus, and lymph nodes, and in non-lymphoid organs, such as the brain, heart, and kidney. In lymphoid organs, Sema4D is abundantly expressed on resting T cells but only weakly expressed on resting B cells and antigen-presenting cells (APCs), such as DCs. Cellular activation increases the surface expression of SEMA4D as well as the generation of soluble SEMA4D (sSEMA4D).

The expression pattern of SEMA4D suggests that it plays an important physiological as well as pathological role in the immune system. SEMA4D has been shown to promote B cell activation, aggregation and survival; enhance CD40-induced proliferation and antibody production; enhance antibody response to T cell dependent antigens; increase T cell proliferation; enhance dendritic cell maturation and ability to stimulate T cells; and is directly implicated in demyelination and axonal degeneration (Shi et al., Immunity 13:633-642 (2000); Kumanogoh et al., J Immunol 169:1175-1181 (2002); and Watanabe et al., J Immunol 167:4321-4328 (2001)). SEMA4D knock out (SEMA4D-/-) mice have provided additional evidence that

SEMA4D plays an important role in both humoral and cellular immune responses. There are no known major abnormalities of non-lymphoid tissues in SEMA4D-/-mice. DCs from the SEMA4D-/- mice have poor allostimulatory ability and show defects in expression of costimulatory molecules, which can be rescued by the addition of sSEMA4D. Mice deficient in SEMA4D (SEMA4D-/-) fail to develop experimental autoimmune encephalomyelitis induced by myelin oligodendrocyte glycoprotein peptide, because myelin oligodendrocyte glycoprotein-specific T cells are poorly generated in the absence of SEMA4D (Kumanogoh et al., J Immunol 169:1175-1181 (2002)). A significant amount of soluble SEMA4D is also detected in the sera of autoimmunity-prone MRL/lpr mice (model of systemic autoimmune diseases such as SLE), but not in normal mice. Further, the levels of sSEMA4D correlate with levels of auto-antibodies and increase with age (Wang et al., Blood 97:3498-3504 (2001)). Soluble SEMA4D has also been shown to accumulate in the cerebral spinal fluid and sera of patients with demyelinating disease, and sSEMA4D induces apoptosis of human pluripotent neural precursors (Dev cells), and both inhibit process extension and induce apoptosis of rat oligodendrocytes in vitro (Giraudon et al., J Immunol 172(2):1246-1255 (2004)). This apoptosis was blocked by an anti-SEMA4D MAb.

### III. Role of Astrocytes and Microglia in Neurodegeneration

Astrocytes are specialized glial cells that perform many essential complex functions in the healthy CNS, including regulation of blood flow, fluid/ion/pH/neurotransmitter homeostasis, synapse formation/function, energy and metabolism, and blood-brain barrier maintenance (Barres B. A. (2008) The mystery and magic of glia: a perspective on their roles in health and disease. Neuron 60:430-440.) Astrocytes respond to CNS injury through a process referred to as reactive astrogliosis, which serves as a major pathological hallmark of neuroinflammatory and neurodegenerative diseases. Increasing evidence points towards the potential of reactive astrogliosis to play either primary or contributing roles in CNS disorders via loss of normal astrocyte functions or gain of abnormal activities. Given their central role in many CNS diseases, there is a significant need to identify and rigorously test new molecular targets that restore normal astrocyte function to effectively slow or even reverse disease progression. There are several potential pathways through which astrocytes can impact CNS diseases.

Microglia are resident immune cells of the brain which derive from a different cell lineage than other cells in the brain. Microglia are highly motile cells, which constantly patrol the brain parenchyma. There are reports that activated microglia contribute to progression of AD. (Clayton, K., “Plaque associated microglia hyper-secrete extracellular vesicles and accelerate tau propagation in a humanized APP mouse model,” Mol. Neurodegen. 12021) 1-6:18; Pascoal, T A, et al., Microglial activation and tau propagate jointly across Braak stages, Nature Med (2021) 27:1592-1599). Conversely, there are also data that suggest that microglial activation is protective rather than pathogenic. This includes genetic evidence that TREM2 variants have both reduced microglial activation and accelerated disease progression. (Leyns C E, Gratuze M, Narasimhan S, Jain N, Koscal L J, Jiang H, Manis M, Colonna M, Lee V M, Ulrich J D, Holtzman D M. “TREM2 function impedes tau seeding in neuritic plaques.” Nat. Neurosci. (2019) 22,1217-1222; Lee S H, Meilandt W J, and Hansen D V. “Trem2 restrains the enhancement of tau accumulation and neurodegeneration by beta amyloid pathology.” Neuron (2021) 109:1283-1301; Lewcock J W, Schlepckow K, Di Paolo G, Tahirovic S, Monroe K M, Haass C. Emerging Microglia Biology Defines Novel Therapeutic Approaches for Alzheimer's Disease. Neuron (2020) 108:801-821).

Despite the apparent contradiction offered by these studies, there is also evidence of microglial heterogeneity at the level of transcriptional patterns, and it has been suggested that microglia can be either protective or pathogenic, depending on their state of activation/inactivation. (Mathys, H. et al. “Temporal tracking of microglia activation in neurodegeneration at single-cell resolution.” Cell Rep. (2017) 21:366-380). Pathogenic activity of microglia may be related to neuroinflammation (Leng F, Edison P. “Neuroinflammation and microglial activation in Alzheimer disease: where do we go from here?” Nat Rev Neurol (2021) 17:157-172) or to promoting the spread of tau seeds. By the same token, secretion of anti-inflammatory cytokines or phagocytosis and degradation of tau seeds could be protective. Some data also suggest that activated microglia can be protective early in neurodegenerative disease but take on a pathogenic phenotype later as the disease progresses. (Mathys, H. et al. (2017); Fan, Z., Brooks, D. J., Okello, A. & Edison, P. “An early and late peak in microglial activation in Alzheimer's disease trajectory.” Brain (2017) 140:792-803).

In neurodegenerative conditions such as Huntington's disease (HD), Alzheimer's disease (AD), Parkinson's disease (PD) and Amyotrophic lateral sclerosis (ALS) for example, neuroinflammation is characterized by a reactive phenotype of glial cells, including both astrocytes and microglia, along with the presence of inflammatory mediators in the brain parenchyma (Ransohoff, (2016), How neuroinflammation contributes to neurodegeneration. Science 353, 777-783; Masgrau et al., (2017), Should we stop saying “glia” and “neuroinflammation”? Trends Mol. Med. 23, 486-500). Studies also have identified changes in microglia, astrocytes, circulating cytokine levels, infiltration of macrophages along with changes in the transcription of genes associated with the control of inflammation, in HD (Crotti and Glass, (2015) “The choreography of neuroinflammation in Huntington's disease.” Trends Immunol. 36, 364-373.; Ransohoff, (2009) Microglial physiology: unique stimuli, specialized responses. Annu. Rev. Immunol. 27, 119-145).

Studies have shown that there is considerable cross talk between astrocytes and microglia. Microglia and astrocytes interact via contact-dependent and secreted factors to modulate their function during normal health and in disease. Liddelow et al. demonstrated that microglia can activate astrocytes (Liddelow S. A. et al., “Neurotoxic reactive astrocytes are induced by activated microglia.” Nature (2017) 541:481-487), while Lian et al. demonstrated that astrocytes can activate microglia (“Astrocyte— microglia cross talk through complement activation modulates amyloid pathology in mouse models of Alzheimer's disease.” J. Neurosci. (2016) 36:577-589). Recently, it was demonstrated that microglia express SEMA4D and are activated through interaction with astrocytes expressing plexin B1/B2 receptors to promote inflammation and CNS pathology. (Clark et al., “Barcoded viral tracing of single-cell interactions in central nervous system inflammation,” 2021. Science 372, eabf1230). Signaling pathways controlled by SEMA4D-Plexin-B1 and SEMA4D-Plexin-B2 as mediators of microglia-astrocyte interactions that promote CNS pathogenesis were identified by Clark et al. (Science 372 eabf1230). Thus, it appears that the SEMA4D/plexin-B1/B2 pathway can activate both astrocytes and microglia.

It is known that neurons upregulate SEMA4D during neurodegenerative disease progression in both HD and AD (Evans EE, Fisher T, Mishra V et al., “REGULATION OF GLIAL CELL ACTIVATION AND NEURODEGENERATION BY ANTI-SEMAPHORIN 4D ANTIBODY PEPINEMAB, POTENTIAL TREATMENT FOR ALZHEIMER'S AND HUNTINGTON'S DISEASE.” AAT-AD/PD™ 2020 abstract) and that astrocytes express plexinB1/B2 receptors (Clark et al., Science 372 eabf1230). Given the intimate association of astrocytes and neurons in the CNS, it is likely that the upregulation of SEMA4D can trigger astrocyte activation. These same activated astrocytes might subsequently activate microglia that also express SEMA4D. In view of the evidence of different roles for astrocytes and microglia depending on their state of activation, there is a significant need to identify the stage(s) of neurodegenerative diseases that are most receptive to treatment with an agent that can reduce the pathogenic effects of these cells while limiting interference with their protective effects.

### IV. Anti-SEMA4D Antibodies

Antibodies that bind SEMA4D have been described in the art. See, for example, U.S. Pat. No. 8,496,938, US Publ. Nos. 2008/0219971 A1, US 2010/0285036 A1, US 2006/0233793 A1, and US 2021/0032329A1, International Patent Applications WO 93/14125, WO 2008/100995, and WO 2010/129917, and Herold et al., Int. Immunol. 7(1): 1-8 (1995), each of which is herein incorporated in its entirety by reference.

In certain embodiments, the antibody blocks the interaction of SEMA4D with one or more of its receptors, e.g., Plexin-Bl or Plexin-B2. Anti-SEMA4D antibodies having these properties can be used in the methods provided herein. Antibodies that can be used include but are not limited to MAbs VX15/2503 (Pepinemab), 67, 76 and D2517 and antigen-binding fragments, variants, or derivatives thereof which are fully described in US 2010/0285036 A1 and US 2001/0032329 A1. Additional antibodies which can be used in the methods provided herein include the BD16 and BB18 antibodies described in US 2006/0233793 A1 as well as antigen-binding fragments, variants, or derivatives thereof; or any of MAb 301, MAb 1893, MAb 657, MAb 1807, MAb 1656, MAb 1808, Mab 59, MAb 2191, MAb 2274, MAb 2275, MAb 2276, MAb 2277, MAb 2278, MAb 2279, MAb 2280, MAb 2281, MAb 2282, MAb 2283, MAb 2284, and MAb 2285, as well as any fragments, variants or derivatives thereof as described in US 2008/0219971 A1. In certain embodiments an anti-SEMA4D antibody for use in the methods provided herein binds human, murine, or both human and murine SEMA4D. Also useful are antibodies which bind to the same epitope as any of the aforementioned antibodies and/or antibodies which competitively inhibit any of the aforementioned antibodies.

In certain embodiments, an anti-SEMA4D antibody or antigen-binding fragment, variant, or derivative thereof useful in the methods provided herein has an amino acid sequence that has at least about 80%, about 85%, about 88%, about 89%, about 90%, about 91%, about 92%, about 93%, about 94%, or about 95% sequence identity to the amino acid sequence for a reference anti-SEMA4D antibody molecule, for example those described above. In a further embodiment, the binding molecule shares at least about 96%, about 97%, about 98%, about 99%, or 100% sequence identity to a reference antibody.

In another embodiment, an anti-SEMA4D antibody or antigen-binding fragment, variant, or derivative thereof useful in the methods provided herein comprises, consists essentially of, or consists of an immunoglobulin heavy chain variable domain (VH domain), where at least one of the CDRs of the VH domain has an amino acid sequence that is at least about 80%, about 85%, about 90%, about 95%, about 96%, about 97%, about 98%, about 99%, or identical to CDR1, CDR2 or CDR3 of SEQ ID NO: 9, 10, or 41.

In another embodiment, an anti-SEMA4D antibody or antigen-binding fragment, variant, or derivative thereof useful in the methods provided herein comprises, consists essentially of, or consists of an immunoglobulin heavy chain variable domain (VH domain), where at least one of the CDRs of the VH domain has an amino acid sequence that is at least about 80%, about 85%, about 90%, about 95%, about 96%, about 97%, about 98%, about 99%, or identical to SEQ ID NO: 6, SEQ ID NO: 7, SEQ ID NO: 8, SEQ ID NO: 42, SEQ ID NO: 43, or SEQ ID NO: 44.

In another embodiment, an anti-SEMA4D antibody or antigen-binding fragment, variant, or derivative thereof useful in the methods provided herein comprises, consists essentially of, or consists of an immunoglobulin heavy chain variable domain (VH domain), where at least one of the CDRs of the VH domain has an amino acid sequence identical, except for 1, 2, 3, 4, or 5 conservative amino acid substitutions, to SEQ ID NO: 6, SEQ ID NO: 7, SEQ ID NO: 8, SEQ ID NO: 42, SEQ ID NO: 43, or SEQ ID NO: 44.

In another embodiment, an anti-SEMA4D antibody or antigen-binding fragment, variant, or derivative thereof useful in the methods provided herein comprises, consists essentially of, or consists of a VH domain that has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 9 , SEQ ID NO: 10, or SEQ ID NO: 41 wherein an anti-SEMA4D antibody comprising the encoded VH domain specifically or preferentially binds to SEMA4D.

In another embodiment, an anti-SEMA4D antibody or antigen-binding fragment, variant, or derivative thereof useful in the methods provided herein comprises, consists essentially of, or consists of an immunoglobulin light chain variable domain (VL domain), where at least one of the CDRs of the VL domain has an amino acid sequence that is at least about 80%, about 85%, about 90%, about 95%, about 96%, about 97%, about 98%, about 99%, or identical to CDR1, CDR2 or CDR3 of SEQ ID NO: 17, 18, or SEQ ID NO: 45.

In another embodiment, an anti-SEMA4D antibody or antigen-binding fragment, variant, or derivative thereof useful in the methods provided herein comprises, consists essentially of, or consists of an immunoglobulin light chain variable domain (VL domain), where at least one of the CDRs of the VL domain has an amino acid sequence that is at least about 80%, about 85%, about 90%, about 95%, about 96%, about 97%, about 98%, about 99%, or identical to SEQ ID NO: 14, SEQ ID NO: 15, SEQ ID NO: 16, SEQ ID NO; 46, SEQ ID NO: 47, or SEQ ID NO: 48.

In another embodiment, an anti-SEMA4D antibody or antigen-binding fragment, variant, or derivative thereof useful in the methods provided herein comprises, consists essentially of, or consists of an immunoglobulin light chain variable domain (VL domain), where at least one of the CDRs of the VL domain has an amino acid sequence identical, except for 1, 2, 3, 4, or 5 conservative amino acid substitutions, to SEQ ID NO: 14, SEQ ID NO: 15, SEQ ID NO: 16, SEQ ID NO: 46, SEQ ID NO: 47, or SEQ ID NO: 48.

In a further embodiment, an anti-SEMA4D antibody or antigen-binding fragment, variant, or derivative thereof useful in the methods provided herein comprises, consists essentially of, or consists of a VL domain that has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 17, SEQ ID NO: 18, or SEQ ID NO: 45 wherein an anti-SEMA4D antibody comprising the encoded VL domain specifically or preferentially binds to SEMA4D.

In a further embodiment, an anti-SEMA4D antibody or antigen-binding fragment, variant, or derivative thereof useful in the methods provided herein comprises, consists essentially of, or consists of a VL and VH domain wherein each has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 17 and SEQ ID NO: 9, respectively, SEQ ID NO: 18 and SEQ ID NO: 10, respectively, or SEQ ID NO: 45 and SEQ ID NO: 41, respectively, wherein an anti-SEMA4D antibody comprising the encoded VL domain specifically or preferentially binds to SEMA4D.

Also included for use in the methods provided herein are polypeptides encoding anti-SEMA4D antibodies, or antigen-binding fragments, variants, or derivatives thereof as described herein, polynucleotides encoding such polypeptides, vectors comprising such polynucleotides, and host cells comprising such vectors or polynucleotides, all for producing anti-SEMA4D antibodies, or antigen-binding fragments, variants, or derivatives thereof for use in the methods described herein.

Suitable biologically active variants of the anti-SEMA4D antibodies of the disclosure can be used in the methods of the present disclosure. Such variants will retain the desired binding properties of the parent anti-SEMA4D antibody. Methods for making antibody variants are generally available in the art.

Methods for mutagenesis and nucleotide sequence alterations are well known in the art. See, for example, Walker and Gaastra, eds. (1983) Techniques in Molecular Biology (MacMillan Publishing Company, New York); Kunkel, Proc. Natl. Acad. Sci. USA 82:488-492 (1985); Kunkel et al., Methods Enzymol. 154:367-382 (1987); Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest can be found in the model of Dayhoff et al. (1978) in Atlas of Protein Sequence and Structure (Natl. Biomed. Res. Found., Washington, D.C.), pp. 345-352, herein incorporated by reference in its entirety. The model of Dayhoff et al. uses the Point Accepted Mutation (PAM) amino acid similarity matrix (PAM 250 matrix) to determine suitable conservative amino acid substitutions. In certain embodiments, conservative substitutions, such as exchanging one amino acid with another having similar properties can be used. Examples of conservative amino acid substitutions as taught by the PAM 250 matrix of the Dayhoff et al. model include, but are not limited to, Gly↔Ala, Val↔Ile↔Leu, Asp↔Glu, Lys↔Arg, Asn↔Gln, and Phe↔Trp↔Tyr.

In constructing variants of the anti-SEMA4D binding molecule, e.g., an antibody or antigen-binding fragment thereof, polypeptides of interest, modifications are made such that variants continue to possess the desired properties, e.g., being capable of specifically binding to a SEMA4D, e.g., human, murine, or both human and murine SEMA4D, e.g., expressed on the surface of or secreted by a cell and having SEMA4D blocking activity, as described herein. Obviously, any mutations made in the DNA encoding the variant polypeptide must not place the sequence out of reading frame and in certain embodiments will not create complementary regions that could produce secondary mRNA structure. See EP Patent Application Publication No. 75,444.

Methods for measuring binding specificity of an anti-SEMA4D binding molecule, e.g., an antibody or antigen-binding fragment, variant, or derivative thereof, include, but are not limited to, standard competitive binding assays, assays for monitoring immunoglobulin secretion by T cells or B cells, T cell proliferation assays, apoptosis assays, ELISA assays, and the like. See, for example, such assays disclosed in WO 93/14125; Shi et al., Immunity 13:633-642 (2000); Kumanogoh et al., J Immunol 169:1175-1181 (2002); Watanabe et al., J Immunol 167:4321-4328 (2001); Wang et al., Blood 97:3498-3504 (2001); and Giraudon et al., J Immunol 172(2):1246-1255 (2004), all of which are herein incorporated by reference.

When discussed herein whether any particular polypeptide, including the constant regions, CDRs, VH domains, or VL domains disclosed herein, is at least about 65%, about 70%, about 75%, about 80%, about 85%, about 90%, about 91%, about 92%, about 93%, about 94%, about 95%, about 96%, about 97%, about 98%, about 99%, or even about 100% identical to another polypeptide, the % identity can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman (1981) Adv. Appl. Math. 2:482-489, to find the best segment of homology between two sequences. When using BESTFIT or any other sequence alignment program to determine whether a particular sequence is, for example, 95% identical to a reference sequence according to the present disclosure, the parameters are set, of course, such that the percentage of identity is calculated over the full length of the reference polypeptide sequence and that gaps in homology of up to 5% of the total number of amino acids in the reference sequence are allowed.

For purposes of the present disclosure, percent sequence identity can be determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant can, for example, differ from a reference anti-SEMA4D antibody (e.g., MAb VX15/2503 (Pepinemab), 67, 76, or D2517) by as few as 1 to 15 amino acid residues, as few as 1 to 10 amino acid residues, such as 6-10, as few as 5, as few as 4, 3, 2, or even 1 amino acid residue.

Percentage of “sequence identity” can also be determined by comparing two optimally aligned sequences over a comparison window. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap drop-off (50), expect value (10) and any other required parameter including but not limited to matrix option.

The constant region of an anti-SEMA4D antibody can be mutated to alter effector function in a number of ways. For example, see U.S. Pat. No. 6,737,056B1 and U.S. Patent Application Publication No. 2004/0132101A1, which disclose Fc mutations that optimize antibody binding to Fc receptors.

In certain anti-SEMA4D antibodies or fragments, variants or derivatives thereof useful in the methods provided herein, the Fc portion can be mutated to decrease effector function using techniques known in the art. For example, the deletion or inactivation (through point mutations or other means) of a constant region domain can reduce Fc receptor binding of the circulating modified antibody thereby increasing tumor localization. In other cases, constant region modifications consistent with the instant disclosure moderate complement binding and thus reduce the serum half-life. Yet other modifications of the constant region can be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, can easily be measured and quantified using well known immunological techniques without undue experimentation. Anti-SEMA4D antibodies for use in the methods provided herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative can contain one or more non-classical amino acids.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an anti-SEMA4D polypeptide, to block SEMA4D interaction with its receptor, or to alleviate symptoms associated with a neurodegenerative disorder in a patient).

For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage or improve a hybridoma's antibody production. Alternatively, non-neutral missense mutations can alter an antibody's ability to bind antigen. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein, (e.g., ability to immunospecifically bind at least one epitope of a SEMA4D polypeptide) can be determined using techniques described herein or by routinely modifying techniques known in the art.

In certain embodiments, the anti-SEMA4D antibodies for use in the methods provided herein comprise at least one optimized complementarity-determining region (CDR). By “optimized CDR” is intended that the CDR has been modified and optimized to improve binding affinity and/or anti-SEMA4D activity that is imparted to an anti-SEMA4D antibody comprising the optimized CDR. “Anti-SEMA4D activity” or “SEMA4D blocking activity” can include activity which modulates one or more of the following activities associated with SEMA4D: B cell activation, aggregation and survival; CD40-induced proliferation and antibody production; antibody response to T cell dependent antigens; T cell or other immune cell proliferation; dendritic cell maturation; demyelination and axonal degeneration; apoptosis of pluripotent neural precursors and/or oligodendrocytes; induction of endothelial cell migration; inhibition of spontaneous monocyte migration; binding to cell surface Plexin-B1 or other receptor, or any other activity associated with soluble SEMA4D or SEMA4D that is expressed on the surface of SEMA4D+cells. Anti-SEMA4D activity can also be attributed to a decrease in incidence or severity of diseases associated with SEMA4D expression, including, but not limited to, certain types of cancers including lymphomas, autoimmune diseases, inflammatory diseases including central nervous system (CNS) and peripheral nervous system (PNS) inflammatory diseases, transplant rejections, and invasive angiogenesis. Examples of optimized antibodies based on murine anti-SEMA4D MAbs BD16 and BB18, were described in US Publ. No. 2008/0219971 A1, International Patent Application WO 93/14125 and Herold et al., Int. Immunol. 7(1): 1-8 (1995), each of which are herein incorporated by reference in their entirety. The modifications can involve replacement of amino acid residues within the CDR such that an anti-SEMA4D antibody retains specificity for the SEMA4D antigen and has improved binding affinity and/or improved anti-SEMA4D activity.

### V. Determining Neurodegenerative Disease Progression of a Subject

The disclosure generally relates to methods of selecting subjects for treatment, methods for determining whether a subject will benefit from treatment and methods of treating patients (e.g., alleviating symptoms) where the subject has, has been diagnosed with, or is suspected of having a progressive neurodegenerative disorder, e.g., a human patient. The methods comprise determining the state of progression of the neurodegenerative disorder and then administering an antibody which specifically binds to SEMA4D, or an antigen-binding fragment, variant, or derivative thereof if the subject's neurodegenerative disease progression meets one or more predetermined test scores that indicate an early or less severe stage of the neurodegenerative disorder, but one which is sufficiently advanced to be more readily susceptible to a successful treatment outcome, e.g., MCI, mild dementia, or moderate dementia or cognitive impairment.

Many degenerative neurological diseases are characterized by gradual memory loss and cognitive deterioration as well as deterioration of muscle function and in some cases, personality changes. A characteristic of many neurodegenerative diseases is progressive neuronal cell death. (Yuan J, Yankner B A: Apoptosis in the nervous system. Nature 2000;407:802-809). Many drugs for treating neurodegenerative diseases only provide symptomatic improvement and are unable to reverse or slow down cognitive decline in patients, such as with AD for example. Many of the unsuccessful drug development efforts are in large part due to inadequate disease characterization. The scientific and medical community do not yet have a sufficient understanding of the pathophysiology of many of these neurodegenerative diseases, their direct causes, and why or how these diseases can present differently in different patients, or what biochemical pathways and factors influence their progression. Clarification of neurodegenerative disease progression is vital to the development of therapeutic agents that are designed to slow the progression of disease.

In the absence of biomarkers to diagnose neurodegenerative diseases appropriately, when the signs and symptoms of PD, HD and AD, for example, appear, it may be far too late to treat the disease effectively. Thus, promising approaches that could have major benefit if used early enough in the disease process may prove ineffective at advanced disease stages, further elaborating on the urgency for biomarkers.

The disclosure provides methods for applying clinical information regarding a subject's disease state or stage of disease progression to determine whether the subject is likely to benefit from treatment with an anti-SEMA4D antibody, allowing for early intervention. Subjects that are diagnosed with, are suspected of having or have a neurodegenerative disorder can be assessed by any number of validated, standardized tests to determine mental status (e.g., cognitive decline, dementia, etc.) and neurophysiological status (e.g., balance, ataxia, muscle twitching, etc.) to determine progression of the disorder. Neuropsychologic tests generally use pencil-and-paper (e.g., the clock drawing test), question-and-answer, and computerized measures to assess cognition. These tests usually have standardized normative information available for comparison with healthy people in the same age range, often stratified by sex, education, and occasionally race. By performing a comprehensive battery of tests, usually with several tests in each domain, it is possible to establish a profile of strengths and weaknesses, which are considered the cognitive “phenotype,” which helps narrow the differential diagnosis. (Weintraub S, Wicklund A H, Salmon D P. The neuropsychological profile of Alzheimer disease. Cold Spring Harb Perspect Med. 2012;2(4)a006171). Multiple other pieces of information may be integrated, including mood and behavior symptoms, medications, medical comorbidities, family history, medical history, and mitigating factors that may affect testing. These data, collected during the record review and interview, are taken into account in test data interpretation and contribute to the overall characterization of an individual's neurobehavioral status, i.e., the stage or phase of the neurodegenerative disorder.

Some well-known screening measures to assess the neurodegenerative state of a subject include for example the Mini-Mental State Examination [MMSE] (Folstein M F, Folstein S E, McHugh P R. “Mini-mental state”: a practical method for grading the cognitive state of patients for the clinician. J Psychiatr Res. 1975;12(3):189-198), the Montreal Cognitive Assessment [MoCA]) (Nasreddine Z S, Phillips N A, Bedirian V, et al. The Montreal Cognitive Assessment, MoCA: a brief screening tool for mild cognitive impairment. J Am Geriatr Soc. 2005;53(4):695-699), the Unified Huntington's Disease Rating Scale (UHDRS) of which the -Total Functional Capacity (TFC) and Total Motor Score (TMS) are components, Huntington's Disease-Cognitive Assessment Battery (HD-CAB), and Clinical Global Impression of Change (CGIC), and several other rests described herein. Often, more than one screening measure is used to assess the level of neurodegeneration in a patient since combining measures into a composite endpoint that reflects multiple cognitive domains can capture cognitive effects more completely.

The TFC test is specific to HD. TFC total score ranges from 0 to 13 with greater scores indicating higher functioning. The TFC has been partitioned into five stages that indicate levels of disease severity based on functional decline. TFC scores from 11-13 represent stage I (least severe); 7-10, stage II; 3-6, stage III; 1-2, stage IV; and score of 0 is stage V (most severe).

Scores on the MoCA scale range from zero to 30, with a score of 26 and higher generally considered normal functioning. A MoCA score in the range of 19-25 indicates mild cognitive impairment (MCI); a MoCA score in the range of 11-21 indicates Mild AD or mild dementia; and a MoCA score of 6-10 indicates moderate dementia. The MoCA is used for assessing any neurodegenerative disorder with associated cognitive impairment, including HD and AD.

The disclosure provides methods for selecting subjects that are diagnosed with, are suspected of having or have a neurodegenerative disorder for treatment with an anti-SEMA4D antibody of the disclosure and methods for determining whether such a subject will benefit from treatment with an anti-SEMA4D antibody of the disclosure, as well as methods of treating such subjects with an anti-SEMA4D antibody as described herein by first determining (or obtaining) a cognitive impairment assessment score using one or more cognitive impairment tests such as those described herein, e.g., the MoCA and/or TFC score, for the subject and selecting and/or treating those subjects that meet a predetermined value for the particular cognitive test. Subjects that are diagnosed with, are suspected of having or have a neurodegenerative disorder and who are determined by to be in a relatively early stage of the neurodegenerative disorder, e.g., a stage associated with MCI, mild or moderate dementia, such as AD or stage I or stage II HD as determined by cognitive and/or functional screening as described herein are shown herein to be more likely to benefit or have a more robust benefit from treatment with an anti-SEMA4D antibody of the disclosure than subjects in an earlier or later stage of the neurodegenerative disorder. More particularly, such subjects who present with one or more cognitive impairment test scores that is or corresponds to a MoCA score of less than 26, which is indicative of cognitive impairment, such as 11-21, 15-21, 19-21, 19-25 or any score between 11-25 or 10-25 such as 10-21 and/or one or more cognitive impairment test score that is or corresponds to a TFC score of 12 or less, such as from 7-12, 7-11, 7-10, 7-9, 8-10, 8-11, 8-12, 9-12, 9-11, or 10-12 for example can benefit from the administration of an anti-SEMA4D antibody of the disclosure.

Generally, the testing and evaluation of one or more scores, comparisons between scores, evaluation of the scores and treatment decisions can be performed by one or more healthcare providers, healthcare benefits providers, and/or clinical laboratories.

The methods of the disclosure are directed to the use of anti-SEMA4D binding molecules, e.g., antibodies, including antigen-binding fragments, variants, and derivatives thereof, to treat the above-described subset of subjects.

In one embodiment, treatment includes the application or administration of an anti-SEMA4D binding molecule, e.g., an antibody or antigen binding fragment thereof or other biologic or small molecule that binds and neutralizes SEMA4D as described herein to a patient, where the patient has or is suspected of having a neurodegenerative disorder. In another embodiment, treatment is also intended to include the application or administration of a pharmaceutical composition comprising the anti-SEMA4D binding molecule, e.g., an antibody or antigen binding fragment thereof to a patient, where the patient has, or has the risk of developing a neurodegenerative disorder.

The anti-SEMA4D binding molecules, e.g., antibodies or binding fragments thereof as described herein are useful for the treatment of various neurodegenerative disorders, particularly during the window of time (or phase of the disease) as described above. In some embodiments, treatment of a neurodegenerative disorder is intended to induce an improvement in the symptoms associated with the disorder. In other embodiments, treatment of a neurodegenerative disorder is intended to reduce, retard or stop an increase in symptom manifestations. In other embodiments, treatment of a neurodegenerative disorder is intended to inhibit, e.g., suppress, retard, prevent, stop, or reverse a manifestation of symptoms. In other embodiments, treatment of a neurodegenerative disorder is intended to relieve to some extent one or more of the symptoms associated with the disorder. In these situations, the symptoms can be neuropsychiatric symptoms, cognitive symptoms, and/or motor dysfunction. In other embodiments, treatment of a neurodegenerative disorder is intended to reduce morbidity and mortality. In other embodiments, treatment of a neurodegenerative disorder is intended to improve quality of life.

In one embodiment, the disclosure relates to the use of anti-SEMA4D binding molecules, e.g., antibodies or antigen-binding fragments, variants, or derivatives thereof, as a medicament, in particular for use in the treatment of neurodegenerative disorders to improve the symptoms associated with the disorder. In another embodiment, the use of anti-SEMA4D binding molecules delays or stops the manifestation of further symptoms.

In accordance with the methods of the present disclosure, at least one anti-SEMA4D binding molecule, e.g., an antibody or antigen binding fragment, variant, or derivative thereof, or other biologic or small molecule as defined elsewhere herein can be used to promote a positive therapeutic response with respect to the neurodegenerative disorder when administered to the subset of subjects identified herein. A “positive therapeutic response” with respect to the neurodegenerative disorder is intended to include an improvement in the symptoms associated with the disorder. Such positive therapeutic responses are not limited to the route of administration and can comprise administration to the donor, the donor tissue (such as for example organ perfusion), the host, any combination thereof, and the like. In particular, the methods provided herein are directed to inhibiting, preventing, reducing, alleviating, or lessening the progression of a neurodegenerative disorder in a patient. Thus, for example, an improvement in the disorder can be characterized as an absence of clinically observable symptoms, a decrease in the incidence of clinically observable symptoms, or a change in the clinically observable symptoms. Improvement (or worsening) of the disorder can be determined by using the same cognitive assessment test used to diagnose the subject's baseline cognitive state or another cognitive assessment test.

The anti-SEMA4D binding molecules, e.g., antibodies or antigen binding fragments, variants, or derivatives thereof or other biologics or small molecules can be used in combination with at least one or more other treatments for neurodegenerative disorders; where the additional therapy is administered prior to, during, or subsequent to administration of the anti-SEMA4D binding molecule, e.g., antibody or antigen binding fragment, variant, or derivative thereof. Thus, where the combined therapies comprise administration of an anti-SEMA4D binding molecule, e.g., an antibody or antigen binding fragment, variant, or derivative thereof, in combination with administration of another therapeutic agent, the methods of the disclosure encompass coadministration, using separate formulations or a single pharmaceutical formulation, with simultaneous or consecutive administration in either order.

To apply the methods of the disclosure in certain embodiments, screening of a subject suspected of having, diagnosed with or who has a neurodegenerative disorder by any of the tests described herein or another standardized cognitive or functional assessment test, such as the TFC and/or MoCA tests, is obtained before (as a means of selection) or both before and after the administration of a therapy comprising an effective amount of a binding molecule that specifically binds to SEMA4D. In some cases, successive tests scores, can be obtained from the selected patient after therapy has commenced, or after therapy has ceased, e.g., monthly, quarterly, or annually for example, in order to determine the effects of treatment.

In certain aspects of any of the aforementioned methods, the neurodegenerative disorder is selected from a group consisting of Alzheimer's disease, Parkinson's disease, (PD) Huntington's disease, (HD) Down syndrome, ataxia, amyotrophic lateral sclerosis (ALS), frontotemporal dementia (FTD), HIV-related cognitive impairment, CNS Lupus, mild cognitive impairment to moderate dementia, or a combination thereof. In certain aspects of any of the aforementioned methods, the neurodegenerative disorder is Alzheimer's disease or Huntington's disease.

### VI. Pharmaceutical Compositions and Administration Methods

Methods of preparing and administering anti-SEMA4D binding molecules, e.g., antibodies, or antigen-binding fragments, variants, or derivatives thereof to a subject in need thereof are well known to or are readily determined by those skilled in the art. The route of administration of the anti-SEMA4D binding molecule, e.g, antibody, or antigen-binding fragment, variant, or derivative thereof, can be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as being within the scope of the disclosure, an example of a form for administration would be a solution for injection, in particular for intravenous or intraarterial injection or drip. A suitable pharmaceutical composition for injection can comprise a buffer (e.g. acetate, phosphate or citrate buffer), a surfactant (e.g. polysorbate), optionally a stabilizer agent (e.g. human albumin), etc.

As discussed herein, anti-SEMA4D binding molecules, e.g., antibodies, or antigen-binding fragments, variants, or derivatives thereof can be administered in a pharmaceutically effective amount for the in vivo treatment of neurodegenerative disorders. In this regard, it will be appreciated that the disclosed binding molecules can be formulated so as to facilitate administration and promote stability of the active agent. In certain embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of an anti-SEMA4D binding molecule, e.g., an antibody, or antigen-binding fragment, variant, or derivative thereof, shall be held to mean an amount sufficient to achieve effective binding to a target and to achieve a benefit, e.g., improve the symptoms associated with a neurodegenerative disorder.

The pharmaceutical compositions used in this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. In the subject disclosure, pharmaceutically acceptable carriers include, but are not limited to, 0.01-0.1 M, e.g., about 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as, for example, antimicrobials, antioxidants, chelating agents, and inert gases and the like.

Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions used in this disclosure can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of an anti-SEMA4D binding molecule, e.g., antibody, or fragment, variant, or derivative thereof, to be combined with the carrier materials to produce a single dosage form will vary depending upon the host treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, anti-SEMA4D antibodies, or antigen-binding fragments, variants, or derivatives thereof can be administered to a human subject in accordance with the aforementioned methods of treatment in an amount sufficient to produce a therapeutic effect. The anti-SEMA4D antibodies, or antigen-binding fragments, variants or derivatives thereof can be administered to such human in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of anti-SEMA4D binding molecules, e.g., antibodies, or antigen-binding fragments, variants, or derivatives thereof, of the disclosure can be used.

Therapeutically effective doses of the compositions of the present disclosure, for the decrease in the incidence of symptoms, vary depending upon many different factors, including means of administration, target site, physiological state of the patient, other medications administered, and whether treatment is prophylactic or therapeutic. Treatment dosages can be titrated using routine methods known to those of skill in the art to optimize safety and efficacy.

The amount of at least one anti-SEMA4D binding molecule, e.g., antibody or binding fragment, variant, or derivative thereof, to be administered is readily determined by one of ordinary skill in the art without undue experimentation given the present disclosure. Factors influencing the mode of administration and the respective amount of at least one anti-SEMA4D binding molecule, e.g., antibody, antigen-binding fragment, variant or derivative thereof include, but are not limited to, the severity of the disease, the history of the disease, and the age, height, weight, health, and physical condition of the individual undergoing therapy. Similarly, the amount of anti-SEMA4D binding molecule, e.g., antibody, or fragment, variant, or derivative thereof, to be administered will be dependent upon the mode of administration and whether the subject will undergo a single dose or multiple doses of this agent.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevere, Amsterdam); Goldsby et al., eds. (2000) Kuby Immunology (4th ed.; H. Freemand & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlang); Sambrook and Russell (2001) Molecular Cloning: A Laboratory Manual (Cold Spring Harbor Press); Lewin (2003) Genes VIII (Prentice Hall 2003); Harlow and Lane (1988) Antibodies: A Laboratory Manual (Cold Spring Harbor Press); Dieffenbach and Dveksler (2003) PCR Primer (Cold Spring Harbor Press).

All of the references cited above, as well as all references cited herein, are incorporated herein by reference in their entireties.

The following examples are offered by way of illustration and not by way of limitation.

### EXAMPLES

Example 1: Two groups of subjects including HD patients who were tested by TFC and at baseline presented with early manifest disease (with TFC score of 11 or 12-13) were treated monthly with either 20 mg/kg of an anti-SEMA4D antibody (Pepinemab, VX15/2503) or a placebo consisting of the diluent for VX15/2503: a sterile solution of 20 mM sodium acetate, pH 5.4, containing 130 mM sodium chloride and 0.02% polysorbate 80 (no preservatives). The antibody and placebo were administered intravenously.

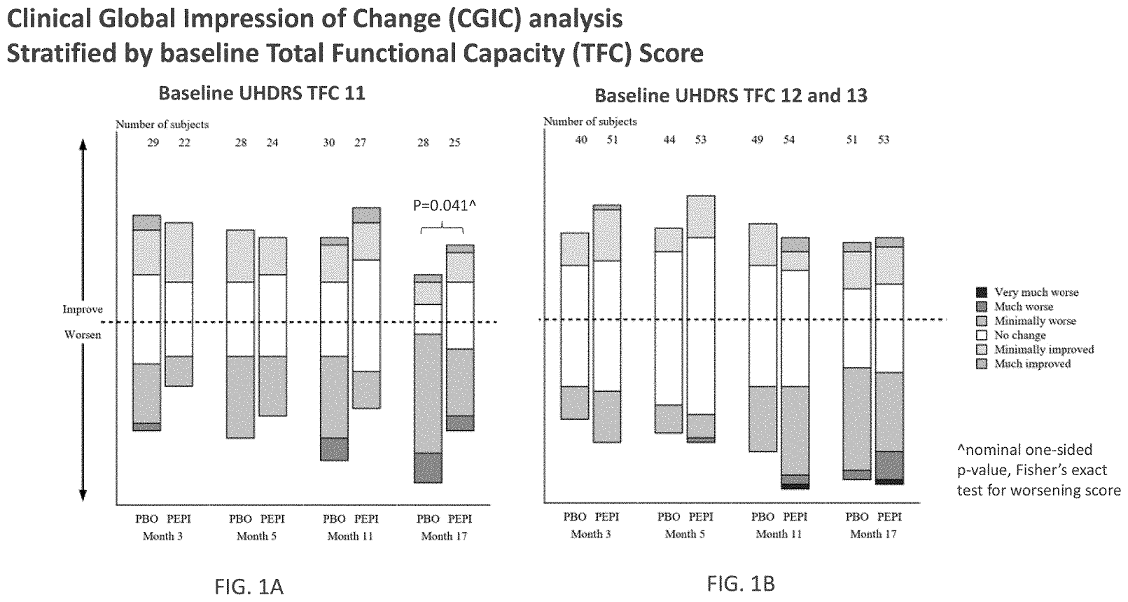

FIG. 1 shows categorical changes in the CGIC at Months 3, 5, 11 and 17 by UHDRS-TFC subgroup (11 and 12-13) in these early manifest patients during the primary analysis period of baseline to month 17. PBO B1=placebo-treated; PEPI B1=VX15/2503 treated. A trend toward treatment benefit was observed in patients who at baseline presented with slightly more advanced disease (TFC11) compared to those with TFC 12-13. Numerically fewer Pepinemab- than placebo-treated subjects (44% versus 71%) with a baseline UHDRS-TFC of 11 had a worsened disease status (light and dark shaded bars below the dotted line) per the CGIC (odds ratio [95% CI]: 0.31 [0.09, 1.12], nominal one-sided p=0.041). For subjects with a UHDRS-TFC score of 11 at baseline, numerically more Pepinemab- than placebo-treated subjects showed no change (white bar) on the CGIC at Month 17. In contrast, no detectable difference in CGIC scores between treatment groups was observed over 18 months of treatment in subjects with a UHDRS-TFC score of 12 or 13 at baseline.

Example 2: Two groups of subjects (early manifest HD patients who had a MoCA score <26 and patients who had a MoCA score ≥26 (cognitively normal) were treated monthly with either 20 mg/kg of an anti-SEMA4D antibody (VX15/2503, Pepinemab) or a placebo consisting of the diluent for VX15/2503: a sterile solution of 20 mM sodium acetate, pH 5.4, containing 130 mM sodium chloride and 0.02% polysorbate 80 (no preservatives). The antibody and placebo were administered intravenously.

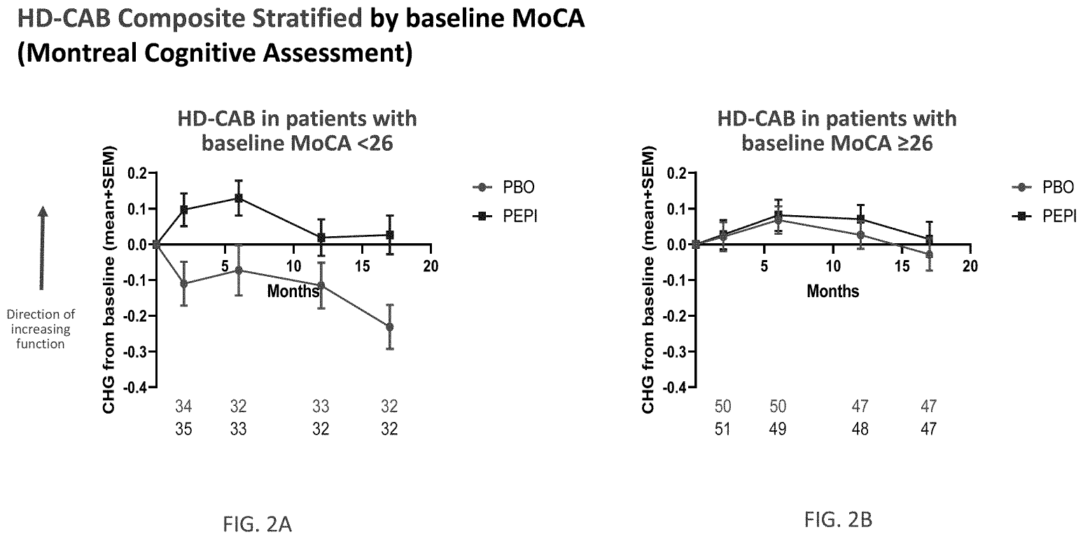

FIG. 2 shows the observed mean changes from baseline in the HD-CAB composite during the primary analysis period in early manifest subjects stratified by baseline MoCA scores, including both MoCA <26 (considered cognitively impaired) (FIG. 2A) and MoCA ≥26 (cognitively normal) (FIG. 2B). VX15/2503 (Pepinemab; PEPI) -treated patients (squares ▪) and placebo (PBO)treated patients (circles ●), number of subjects, n, at each timepoint for each group is listed at the bottom (top row=placebo-treated; bottom row=Pepinemab-treated subjects), and error bars represent SEM. Greater HD-CAB scores represent higher cognitive function. In patients that demonstrated cognitive impairment at baseline, there is a detectable decline in HD-CAB scores in the placebo group, while Pepinemab treatment shows a halt, slowing or reversal of cognitive decline as compared to placebo. In contrast, in patients with normal cognitive functioning at baseline, there is minimal decline in the placebo group over the course of the study, and a treatment difference is not evident.

Example 3: Two groups of subjects (early manifest HD patients who had a MoCA score <26 (considered cognitively impaired) and patients who had a MoCA score ≥26 (cognitively normal) were treated monthly with either 20 mg/kg of an anti-SEMA4D antibody (VX15/2503, Pepinemab) or a placebo consisting of the diluent for VX15/2503: a sterile solution of 20 mM sodium acetate, pH 5.4, containing 130 mM sodium chloride and 0.02% polysorbate 80 (no preservatives). The antibody and placebo were administered intravenously.

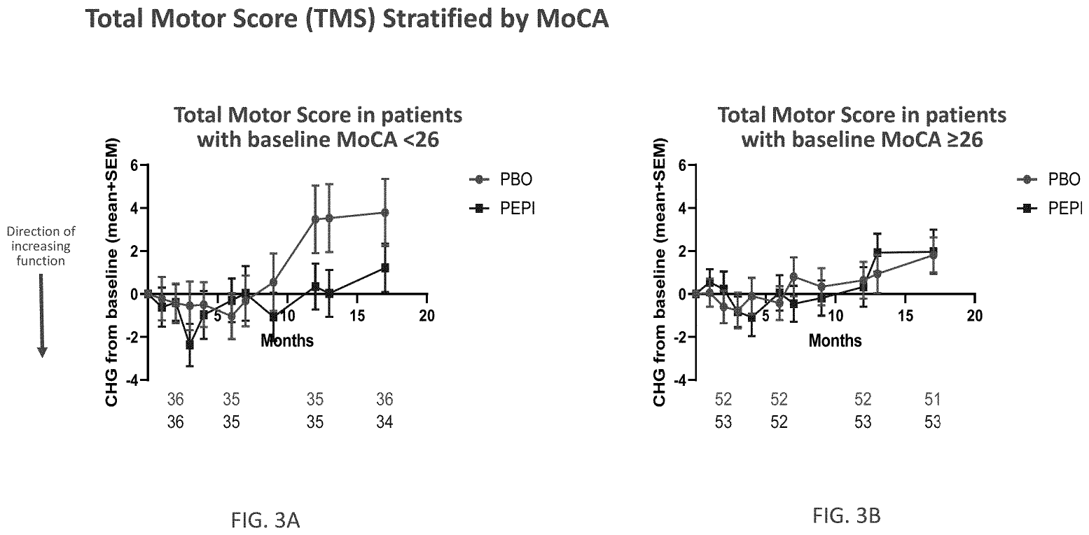

FIG. 3 shows the observed mean changes from baseline in the Total Motor

Score (TMS) during the primary analysis period in early manifest subjects stratified by baseline MoCA scores, including both MoCA <26 (considered cognitively impaired) (FIG. 3A) and MoCA ≥26 (cognitively normal) (FIG. 3B). VX15/2503 (PEPI)-treated patients are indicated by squares (▪) and Placebo (PBO) treated patients are indicated by circles (●), number of subjects, n, at each timepoint for each group are listed at the bottom (top row=placebo-treated; bottom row=Pepinemab-treated subjects), and error bars represent SEM. Lower TMS scores represent better motor function. In patients that demonstrate cognitive impairment at baseline, there is an increase in motor impairment in the placebo group, while Pepimemab treatment shows a slowing of motor impairment as compared to placebo. In contrast, in patients with normal cognitive functioning at baseline, there is minimal change in motor impairment in the placebo group over the course of the study, and the treatment difference is not evident.

The following sequences define antibody D2517 which is described in US Patent Application No. 20210032329, incorporated herein by reference in its entirely:

(SEQ ID NO: 41; D2517VH):
Glu Val Gln Leu Val Glu Ser Gly Gly Gly Leu Val
Gln Pro Gly Gly Ser Leu Arg Leu Ser Cys Ala Ala
Ser Gly Phe Ile Phe Ser Asp Tyr Trp Met Val Trp
Val Arg Gln Ala Pro Gly Lys Gly Leu Glu Tyr Val
Ala His Met Asn Gln Asp Gly Gly Ala Arg Tyr Tyr
Ala Glu Ser Val Arg Gly Arg Phe Thr Ile Ser Arg
Asp Asn Ala Lys Asn Ser Leu Tyr Leu Gln Met Asn
Ser Leu Arg Ala Glu Asp Thr Ala Val Tyr Tyr Cys
Ala Arg Asp Pro Trp Gly Tyr Asp Ser Trp Gly Gln
Gly Thr Leu Val Thr
(SEQ ID NO: 42; D2517 VHCDR1):
Asp Tyr Trp Met Val
(SEQ ID NO: 43; D2517 VH CDR2):
His Met Asn Gln Asp Gly Gly Ala Arg Tyr Tyr
(SEQ ID NO: 44; D2517 VH CDR3):
Asp Pro Trp Gly Tyr
(SEQ ID NO: 45 (D2517 VL):
Ser Tyr Glu Leu Thr Gln Pro Pro Ser Val Ser Val
Ser Pro Gly Gln Thr Ala Ser Ile Thr Cys Ser Gly
Asp Lys Leu Gly Asp Lys Tyr Ala Val Trp Tyr Gln
Gln Lys Pro Gly Gln Ser Pro Val Leu Val Ile Tyr
Gln Asp Ser Lys Arg Pro Ser Gly Ile Pro Glu Arg
Phe Ser Gly Ser Asn Ser Gly Asn Thr Ala Thr Leu
Thr Ile Ser Gly Thr Gln Ala Met Asp Glu Ala Asp
Tyr Tyr Cys Gln Ala Trp Glu Gln Glu Ala Ala Trp
Val Phe Gly Gly Gly Thr Lys Leu
(SEQ ID NO: 46; D2517 VL CDR1):
Ser Gly Asp Lys Leu Gly Asp Lys Tyr Ala Val
(SEQ ID NO: 47; VL CDR2):
Gln Asp Ser Lys Arg Pro Ser
(SEQ ID NO: 48; D2517 VL CDR3):
Gln Ala Trp Glu Gln Glu Ala Ala Trp Val

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims and list of embodiments disclosed herein. Although specific terms are employed herein, they are used in a generic and descriptive sense only and not for purposes of limitation.

What is claimed is:

1. A method of selecting subjects having, determined to have, or suspected of having a neurodegenerative disorder for treatment with an isolated antibody or antigen-binding fragment thereof that specifically binds to semaphorin-4D (SEMA4D), said method comprising:
(i) determining the cognitive and/or functional impairment assessment score for the subject in one or more standard cognitive and/or functional assessment tests; and
(ii) selecting the subject for treatment if the score satisfies a predetermined value indicative of mild cognitive impairment (MCI), mild dementia, moderate cognitive impairment, or Stage I or Stage II Huntington's disease.

2. The method of claim 1, wherein the one or more standard cognitive assessment and/or functional assessment tests are selected from the Montreal Cognitive Assessment (MoCA), the Total Functional Capacity (TFC), Clinical Global Impression of Change (CGIC), and the Clinical Global Impression of Severity (CGIS).

3. The method of claim 1, wherein the one or more standard functional assessment test is the TFC and wherein the functional impairment assessment score for the subject is in the range of 7-12.

4. The method of claim 3, wherein the functional impairment assessment score for the subject is in the range of 8-12 or 8-11.

5. The method of claim 1, wherein the one or more standard cognitive assessment test is the MoCA and wherein the cognitive impairment assessment score for the subject is in the range of from 10-25.

6. The method of claim 5, wherein the MoCA score is in the range of from 19-25.

7. The method of claim 5, wherein the MoCA score is in the range of from 11-21.

8. The method of claim 1, wherein the neurodegenerative disorder is selected from a group consisting of Alzheimer's disease, Parkinson's disease, Huntington's disease, Down syndrome, ataxia, amyotrophic lateral sclerosis (ALS), frontotemporal dementia (FTD), HIV-related cognitive impairment, CNS Lupus, mild cognitive impairment to moderate cognitive impairment, or a combination thereof.

9. The method of claim 8, wherein the neurodegenerative disorder is Alzheimer's disease (AD) or Huntington's disease (HD).

10. The method of claim 1, wherein the antibody or antigen-binding fragment thereof that specifically binds to SEMA4D comprises a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively, or comprises a variable heavy chain (VH) comprising VHCDRs 1-3 comprising SEQ ID NOs 42, 43, and 44, respectively, and a variable light chain (VL) comprising VLCDRs 1-3 comprising SEQ ID NOs 46, 47, and 48, respectively.

11. The method of claim 1, wherein the antibody or antigen binding fragment thereof inhibits SEMA4D binding to its receptor.

12. The method of claim 11, wherein the receptor is selected from Plexin-B1 and Plexin-B2.

13. The method of claim 11, wherein the antibody or antigen binding fragment thereof inhibits SEMA4D-mediated signal transduction.

14. A method of treating a subject having, determined to have, or suspected of having a neurodegenerative disorder, comprising
(i) determining the cognitive and/or functional impairment assessment score for the subject in one or more standard cognitive and/or functional assessment tests; and
(ii) administering a therapeutically effective amount of an isolated antibody or antigen-binding fragment thereof that specifically binds to semaphorin-4D (SEMA4D) if the score satisfies a predetermined value indicative of mild cognitive impairment (MCI), mild dementia, moderate cognitive impairment or Stage I or Stage II Huntington's disease.

15. The method of claim 14, wherein the one or more standard cognitive or functional assessment tests are selected from the Montreal Cognitive Assessment (MoCA), the Total Functional Capacity (TFC), Clinical Global Impression of Change (CGIC), and the Clinical Global Impression of Severity (CGIS).

16. The method of claim 14, wherein the standard functional assessment test is the TFC and wherein the functional impairment assessment score for the subject is in the range of 7-12.

17. The method of claim 15, wherein the functional impairment assessment score for the subject is in the range of 8-12 or 8-11.

18. The method of claim 14, wherein the standard cognitive assessment test is the MoCA and wherein the cognitive impairment assessment score for the subject is in the range of from 10-25.

19. The method of claim 18, wherein the MoCA score is in the range of from 19-25.

20. The method of claim 18, wherein the MoCA score is in the range of from 11-21.

21. The method of claim 14, wherein the neurodegenerative disorder is selected from a group consisting of Alzheimer's disease, Parkinson's disease, Huntington's disease, Down syndrome, ataxia, amyotrophic lateral sclerosis (ALS), frontotemporal dementia (FTD), HIV-related cognitive impairment, CNS Lupus, mild cognitive impairment, or a combination thereof.

22. The method of claim 21, wherein the neurodegenerative disorder is Alzheimer's disease (AD) or Huntington's disease (HD).

23. The method of claim 14, wherein the antibody or antigen-binding fragment thereof that specifically binds to SEMA4D comprises a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively or comprises a variable heavy chain (VH) comprising VHCDRs 1-3 comprising SEQ ID NOs 42, 43, and 44, respectively, and a variable light chain (VL) comprising VLCDRs 1-3 comprising SEQ ID NOs 46, 47, and 48, respectively.

24. The method of claim 14, wherein the antibody or antigen binding fragment thereof inhibits SEMA4D binding to its receptor.

25. The method of claim 24, wherein the receptor is selected from Plexin-B1 and Plexin-B2.

26. The method of claim 24, wherein the antibody or antigen binding fragment thereof inhibits SEMA4D-mediated signal transduction.

27. The method of claim 14, wherein the treatment results in a decrease, reduction, slowing or stopping of the incidence of symptoms associated with the neurodegenerative disorder; a decrease, reduction or lessening of the severity of symptoms associated with the neurodegenerative disorder; or improves the quality of life of the subject.

28. The method of claim 27, wherein the symptoms are selected from neuropsychiatric symptoms, cognitive symptoms, motor dysfunction and any combination thereof.


SEQUENCE LISTING (48 sequences)

SEQ ID NO: 1 - protein, 862 aa, Homo sapiens
MRMCTPIRGLLMALAVMFGTAMAFAPIPRITWEHREVHLVQFHEPDIYNYSALLLSEDKDTLYIGAREAVFAVNALNISEKQHEVYWKVSEDKKAKCAEKGKSKQTECLNYIRVLQPLSATSLYVCGTNAFQPACDHLNLTSFKFLGKNEDGKGRCPFDPAHSYTSVMVDGELYSGTSYNFLGSEPIISRNSSHSPLRTEYAIPWLNEPSFVFADVIRKSPDSPDGEDDRVYFFFTEVSVEYEFVFRVLIPRIARVCKGDQGGLRTLQKKWTSFLKARLICSRPDSGLVFNVLRDVFVLRSPGLKVPVFYALFTPQLNNVGLSAVCAYNLSTAEEVFSHGKYMQSTTVEQSHTKWVRYNGPVPKPRPGACIDSEARAANYTSSLNLPDKTLQFVKDHPLMDDSVTPIDNRPRLIKKDVNYTQIVVDRTQALDGTVYDVMFVSTDRGALHKAISLEHAVHIIEETQLFQDFEPVQTLLLSSKKGNRFVYAGSNSGVVQAPLAFCGKHGTCEDCVLARDPYCAWSPPTATCVALHQTESPSRGLIQEMSGDASVCPDKSKGSYRQHFFKHGGTAELKCSQKSNLARVFWKFQNGVLKAESPKYGLMGRKNLLIFNLSEGDSGVYQCLSEERVKNKTVFQVVAKHVLEVKVVPKPVVAPTLSVVQTEGSRIATKVLVASTQGSSPPTPAVQATSSGAITLPPKPAPTGTSCEPKIVINTVPQLHSEKTMYLKSSDNRLLMSLFLFFFVLFLCLFFYNCYKGYLPRQCLKFRSALLIGKKKPKSDFCDREQSLKETLVEPGSFSQQNGEHPKPALDTGYETEQDTITSKVPTDREDSQRIDDLSARDKPFDVKCELKFADSDADGD

SEQ ID NO: 2 - protein, 861 aa, Mus sp.
  source 1..861: note = Murine sp.
MRMCAPVRGLFLALVVVLRTAVAFAPVPRLTWEHGEVGLVQFHKPGIFNYSALLMSEDKDTLYVGAREAVFAVNALNISEKQHEVYWKVSEDKKSKCAEKGKSKQTECLNYIRVLQPLSSTSLYVCGTNAFQPTCDHLNLTSFKFLGKSEDGKGRCPFDPAHSYTSVMVGGELYSGTSYNFLGSEPIISRNSSHSPLRTEYAIPWLNEPSFVFADVIQKSPDGPEGEDDKVYFFFTEVSVEYEFVFKLMIPRVARVCKGDQGGLRTLQKKWTSFLKARLICSKPDSGLVFNILQDVFVLRAPGLKEPVFYAVFTPQLNNVGLSAVCAYTLATVEAVFSRGKYMQSATVEQSHTKWVRYNGPVPTPRPGACIDSEARAANYTSSLNLPDKTLQFVKDHPLMDDSVTPIDNRPKLIKKDVNYTQIVVDRTQALDGTFYDVMFISTDRGALHKAVILTKEVHVIEETQLFRDSEPVLTLLLSSKKGRKFVYAGSNSGVVQAPLAFCEKHGSCEDCVLARDPYCAWSPAIKACVTLHQEEASSRGWIQDMSGDTSSCLDKSKESFNQHFFKHGGTAELKCFQKSNLARVVWKFQNGELKAASPKYGFVGRKHLLIFNLSDGDSGVYQCLSEERVRNKTVSQLLAKHVLEVKMVPRTPPSPTSEDAQTEGSKITSKMPVASTQGSSPPTPALWATSPRAATLPPKSSSGTSCEPKMVINTVPQLHSEKTVYLKSSDNRLLMSLLLFIFVLFLCLFSYNCYKGYLPGQCLKFRSALLLGKKTPKSDFSDLEQSVKETLVEPGSFSQQNGDHPKPALDTGYETEQDTITSKVPTDREDSQRIDELSARDKPFDVKCELKFADSDADGD

SEQ ID NO: 3 - DNA, 30 bp, synthetic construct
  source 1..30: mol_type = other DNA; note = Polynucleotide anti-CD100 VH CDR1
ggctacagcttcagcgactactacatgcac

SEQ ID NO: 4 - DNA, 51 bp, synthetic construct
  source 1..51: mol_type = other DNA; note = Polynucleotide anti-CD100 VH CDR2
cagattaatcctaccactggcggcgctagctacaaccagaagttcaagggc

SEQ ID NO: 5 - DNA, 27 bp, synthetic construct
  source 1..27: mol_type = other DNA; note = Polynucleotide anti-CD100 VH CDR3
tattactacggcagacacttcgatgtc

SEQ ID NO: 6 - protein, 10 aa, synthetic construct
  source 1..10: note = Polypeptide anti-CD100 VH CDR1
GYSFSDYYMH

SEQ ID NO: 7 - protein, 17 aa, synthetic construct
  source 1..17: note = Polypeptide anti-CD100 VH CDR2
QINPTTGGASYNQKFKG

SEQ ID NO: 8 - protein, 9 aa, synthetic construct
  source 1..9: note = Polypeptide anti-CD100 VH CDR3
YYYGRHFDV

SEQ ID NO: 9 - protein, 118 aa, synthetic construct
  source 1..118: note = Polypeptide anti-CD100 VH 2503
QVQLVQSGAEVKKPGSSVKVSCKASGYSFSDYYMHWVRQAPGQGLEWMGQINPTTGGASYNQKFKGKATITVDKSTSTAYMELSSLRSEDTAVYYCARYYYGRHFDVWGQGTTVTVSS

SEQ ID NO: 10 - protein, 118 aa, synthetic construct
  source 1..118: note = Polypeptide anti-CD100 VH 67
QVQLQQSGPELVKPGASVKISCKASGYSFSDYYMHWVKQSPENSLEWIGQINPTTGGASYNQKFKGKATLTVDKSSSTAYMQLKSLTSEESAVYYCTRYYYGRHFDVWGQGTTVTVSS

SEQ ID NO: 11 - DNA, 45 bp, synthetic construct
  source 1..45: mol_type = other DNA; note = Polynucleotide anti-CD100 VL CDR1
aaggccagccaaagcgtggattatgatggcgatagctatatgaac

SEQ ID NO: 12 - DNA, 21 bp, synthetic construct
  source 1..21: mol_type = other DNA; note = Polynucleotide anti-CD100 VL CDR2
gctgcatccaatctggaaagc

SEQ ID NO: 13 - DNA, 27 bp, synthetic construct
  source 1..27: mol_type = other DNA; note = Polynucleotide anti-CD100 VL CDR3
cagcaaagcaatgaggatccctacacc

SEQ ID NO: 14 - protein, 15 aa, synthetic construct
  source 1..15: note = Polypeptide anti-CD100 VL CDR1
KASQSVDYDGDSYMN

SEQ ID NO: 15 - protein, 7 aa, synthetic construct
  source 1..7: note = Polypeptide anti-CD100 VL CDR2
AASNLES

SEQ ID NO: 16 - protein, 9 aa, synthetic construct
  source 1..9: note = Polypeptide anti-CD100 VL CDR3
QQSNEDPYT

SEQ ID NO: 17 - protein, 111 aa, synthetic construct
  source 1..111: note = Polypeptide anti-CD100 VL 2503
DIVMTQSPDSLAVSLGERATINCKASQSVDYDGDSYMNWYQQKPGQPPKLLIYAASNLESGVPDRFSGSGSGTDFTLTISSLQAEDVAVYYCQQSNEDPYTFGQGTKLEIK

SEQ ID NO: 18 - protein, 111 aa, synthetic construct
  source 1..111: note = Polypeptide anti-CD100 VL 67
DIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 19 - DNA, 354 bp, synthetic construct
  source 1..354: mol_type = other DNA; note = Polynucleotide anti-CD100 VH 2503
caggtgcagctggtgcagagcggcgctgaggtgaagaagcctggcagcagcgtgaaggtctcctgcaaggctagcggctacagcttcagcgactactacatgcactgggtgagacaggcccctggccaaggcctggagtggatgggccagattaatcctaccactggcggcgctagctacaaccagaagttcaagggcaaggccaccattaccgtggacaaaagcaccagcacagcctacatggagctgagcagcctgagaagcgaggacaccgccgtgtattactgtgccagatattactacggcagacacttcgatgtctggggccaaggcaccacggtcaccgtctcttca

SEQ ID NO: 20 - DNA, 354 bp, synthetic construct
  source 1..354: mol_type = other DNA; note = Polynucleotide anti-CD100 VH 67
caggtccagctgcagcagtctggacctgagctggtgaagcctggggcttcagtgaagatatcctgcaaggcttctggttactcattcagtgactactacatgcactgggtgaagcaaagtcctgaaaatagtcttgagtggattggacagattaatcctaccactgggggtgctagctacaaccagaagttcaagggcaaggccacattaactgtagataaatcctccagcacagcctacatgcagctcaagagcctgacatctgaagagtctgcagtctattactgtacaagatattactacggtagacacttcgatgtctggggccaagggaccacggtcaccgtttcctca

SEQ ID NO: 21 - DNA, 333 bp, synthetic construct
  source 1..333: mol_type = other DNA; note = Polynucleotide anti-CD100 VL 2503
gacatcgtgatgacccagagcccagacagcctggctgtgagcctgggcgagagggccaccatcaactgcaaggccagccaaagcgtggattatgatggcgatagctatatgaactggtaccagcagaaaccaggccagcctcctaagctgctgatttacgctgcatccaatctggaaagcggcgtgcctgacagattcagcggcagcggcagcggcacagatttcactctgaccatcagcagcctgcaggctgaagatgtggcagtgtattactgtcagcaaagcaatgaggatccctacaccttcggccaagggaccaagctcgagatcaaa

SEQ ID NO: 22 - DNA, 333 bp, synthetic construct
  source 1..333: mol_type = other DNA; note = Polynucleotide anti-CD100 VL 67
gacattgtgatgacccagtctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctcgagatcaaa

SEQ ID NO: 23 - protein, 118 aa, synthetic construct
  source 1..118: note = Polypeptide anti-CD100 VH 76
QVQLQQSGAELAKPGASVKMSCKASGYTFTRYWMHWVKQRPGQGLEWIGYINPSTGYSDYNQKFKDKATLTADKSSSTAYMQLSSLTSEDSAVYYCARDPYGWTMDSWGQGTLVTVSS

SEQ ID NO: 24 - protein, 10 aa, synthetic construct
  source 1..10: note = Polypeptide anti-CD100 VH 76 CDR1
GYTFTRYWMH

SEQ ID NO: 25 - protein, 17 aa, synthetic construct
  source 1..17: note = Polypeptide anti-CD100 VH 76 CDR2
YINPSTGYSDYNQKFKD

SEQ ID NO: 26 - protein, 9 aa, synthetic construct
  source 1..9: note = Polypeptide anti-CD100 VH 76 CDR3
DPYGWTMDS

SEQ ID NO: 27 - protein, 107 aa, synthetic construct
  source 1..107: note = Polypeptide anti-CD100 VL 76
DIQMTQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLEIK

SEQ ID NO: 28 - protein, 11 aa, synthetic construct
  source 1..11: note = Polypeptide anti-CD100 VL 76 CDR1
HASQNINVWLS

SEQ ID NO: 29 - protein, 7 aa, synthetic construct
  source 1..7: note = Polypeptide anti-CD100 VL 76 CDR2
KASNLHT

SEQ ID NO: 30 - protein, 9 aa, synthetic construct
  source 1..9: note = Polypeptide anti-CD100 VL 76 CDR3
QQGQSYPYT

SEQ ID NO: 31 - DNA, 354 bp, synthetic construct
  source 1..354: mol_type = other DNA; note = Polynucleotide anti-CD100 VH 76
caggtccagctgcagcagtctggggctgaactggcaaaacctggggcctcagtgaagatgtcctgcaaggcttctggctacacctttactaggtactggatgcactgggtaaaacagaggcctggacagggtctggaatggattggatacattaatcctagcactggttattctgattacaatcagaagttcaaggacaaggccacattgactgcagacaaatcctccagcacagcctacatgcaactgagcagcctgacatctgaggactctgcagtctattactgtgcaagagacccctacggctggactatggactcctggggccaagggactctggtcaccgtctcctca

SEQ ID NO: 32 - DNA, 30 bp, synthetic construct
  source 1..30: mol_type = other DNA; note = Polynucleotide anti-CD100 VH 76 CDR1
ggctacacctttactaggtactggatgcac

SEQ ID NO: 33 - DNA, 51 bp, synthetic construct
  source 1..51: mol_type = other DNA; note = Polynucleotide anti-CD100 VH 76 CDR2
tacattaatcctagcactggttattctgattacaatcagaagttcaaggac

SEQ ID NO: 34 - DNA, 27 bp, synthetic construct
  source 1..27: mol_type = other DNA; note = Polynucleotide anti-CD100 VH 76 CDR3
gacccctacggctggactatggactcc

SEQ ID NO: 35 - DNA, 321 bp, synthetic construct
  source 1..321: mol_type = other DNA; note = Polynucleotide anti-CD100 VL 76
gacatccagatgacccagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatccgtacacgttcggaggggggaccaagctcgagatcaaa

SEQ ID NO: 36 - DNA, 33 bp, synthetic construct
  source 1..33: mol_type = other DNA; note = Polynucleotide anti-CD100 VL 76 CDR1
catgccagtcagaacattaatgtttggttaagc

SEQ ID NO: 37 - DNA, 21 bp, synthetic construct
  source 1..21: mol_type = other DNA; note = Polynucleotide anti-CD100 VL 76 CDR2
aaggcttccaacttgcacaca

SEQ ID NO: 38 - DNA, 27 bp, synthetic construct
  source 1..27: mol_type = other DNA; note = Polynucleotide anti-CD100 VL 76 CDR3
caacagggtcaaagttatccgtacacg

SEQ ID NO: 39 - protein, 113 aa, synthetic construct
  source 1..113: note = 2282 VL domain
DIVMTQSPLSLPVSPGEPATINCKSSQSLFNSGNQKNYLAWYLQKPGQPPKLLIYGASTRESGVPDRFSGSGSGTDFTLTISSLQAEDVAVYYCQNDHTYPYTFGQGTKLEIK

SEQ ID NO: 40 - protein, 122 aa, synthetic construct
  source 1..122: note = 2282 VH domain
QVQLVQSGAEVKKPGSSVKVSCTASGYTFTDYYMDWVRQAPGQGLEWMGRVNPYHGYATYNQKFKGRVTITADKSTSTAYMELSSLRSEDTAVYYCAREENSYDGYYGMDYWGQGTLVTVSS

SEQ ID NO: 41 - protein, 113 aa, synthetic construct
EVQLVESGGGLVQPGGSLRLSCAASGFIFSDYWMVWVRQAPGKGLEYVAHMNQDGGARYYAESVRGRFTISRDNAKNSLYLQMNSLRAEDTAVYYCARDPWGYDSWGQGTLVT

SEQ ID NO: 42 - protein, 5 aa, synthetic construct
DYWMV

SEQ ID NO: 43 - protein, 11 aa, synthetic construct
HMNQDGGARYY

SEQ ID NO: 44 - protein, 5 aa, synthetic construct
DPWGY

SEQ ID NO: 45 - protein, 104 aa, synthetic construct
SYELTQPPSVSVSPGQTASITCSGDKLGDKYAVWYQQKPGQSPVLVIYQDSKRPSGIPERFSGSNSGNTATLTISGTQAMDEADYYCQAWEQEAAWVFGGGTKL

SEQ ID NO: 46 - protein, 11 aa, synthetic construct
SGDKLGDKYAV

SEQ ID NO: 47 - protein, 7 aa, synthetic construct
QDSKRPS

SEQ ID NO: 48 - protein, 10 aa, synthetic construct
QAWEQEAAWV